(19) United States
(12) Patent Application Publication
(10) Pub. No.: US 2025/0135182 A1
(43) Pub. Date: May 1, 2025

(54) System, Device and Method Having Patient-Specific Myocardial Performance Modeling for Cardiovascular Performance Monitoring and Control

(71) Applicant: Jonathan Grinstein, Chicago, IL (US)

(72) Inventor: Jonathan Grinstein, Chicago, IL (US)

(21) Appl. No.: 18/927,534

(22) Filed: Oct. 25, 2024

Related U.S. Application Data
(60) Provisional application No. 63/545,908, filed on Oct. 26, 2023.

(51) Int. Cl. A61M 60/531; A61M 5/142; A61M 5/172; A61M 25/00; A61M 60/178

(52) U.S. Cl. CPC A61M 60/531; A61M 5/14244; A61M 5/1723; A61M 60/178; A61M 2025/0001; A61M 2205/18; A61M 2205/502; A61M 2230/30

(57) ABSTRACT

A cardiac monitoring control system including a display device, a detection system to periodically detect parameters at a load, a delivery system coupled to the load to deliver a treatment to the load, and a controller comprising a processor and a memory storing instructions. When executed, the instructions cause the processor to receive signals indicative of the parameters periodically detected at the load transmitted by the detection system, and iteratively generate a user-specific ventriculo-arterial coupling (VAC) ratio based on the signals received. The display device animates the VAC ratio dynamically, and the delivery system is activated to deliver a user-specific treatment updated dynamically based on the VAC ratio.

### RELATED APPLICATION

The present application claims priority to U.S. Provisional Patent Application No. 63/545,908, filed Oct. 26, 2023, which is hereby incorporated by reference in its entirety.

### BACKGROUND

Enabling real-time and accurate determination of an organ's performance is central to the assessment of an organ's function and potential treatment or therapies. Various efforts to improve the real-time and accurate assessment of cardiovascular performance, whether in treatment of cardiovascular disease or in the context of physiological performance in healthy individuals, have been lacking.

Hemodynamic assessment has played a vital role in risk stratification in heart failure. Indeed, timely referral for advanced therapies is of the upmost importance for patients with various cardiovascular diseases, e.g., advanced heart failure and cardiogenic shock, given the rapid and unpredictable progression of the cardiovascular disease. However, a fundamental limitation of hemodynamic data is the reliance on thresholds for each parameter or metric. These parameters are sometimes arbitrary (e.g., pulmonary capillary wedge pressure or PCWP>15, or 18 mmHg, or cardiac index or CI<2.2, 2.0, or 1.8 L/min/m²) and often poorly defined across the continuity of heart failure.

For certain other parameters, discrete thresholds that are statistical in nature (not physiological thresholds) and are thresholds defined using receiver-operator-characteristic analysis from a specific patient population that may or may not be representative of a given individual, e.g., a patient under a provider's care or a performance athlete. Furthermore, these statistical thresholds vary based on the disease acuity of the specific patient population being studied. As such, the statistical thresholds are rendered arbitrary and poorly defined parameters.

Routine use of continuous hemodynamic monitoring, for both risk assessment and management, has also had a tumultuous history. Pulmonary artery catheters (PAC) have been utilized for real-time hemodynamic monitoring dating back to the 1970s. However, their use abruptly declined following the publication of the ESCAPE trial in the early 2000s, which failed to show a survival benefit with routine PAC use. More recently, there has been a resurgence of PAC use following emerging data that a complete invasive hemodynamic assessment may confer some survival benefit.

Despite increased attention on continuous hemodynamic monitoring as a standard hemodynamic assessment, its focus on measuring intracardiac filling pressures and cardiac output has had variable prognostic performance. While elevated filling pressures routinely confer a poor prognosis, a low cardiac output or cardiac index have had more inconsistent or inconstant prognostic certainty.

To overcome the limitations of standard hemodynamic parameters, advanced hemodynamic parameters were derived to better reflect the interaction of loading conditions and cardiac performance. The left-sided advanced parameters of cardiac power output (CPO), aortic pulsatility index (API) and left ventricular stroke work index (LVSWI) have improved prognostic performance over standard hemodynamic parameters. Similarly, the right-sided advanced parameters of pulmonary artery pulsatility index (PAPI) and right ventricular stroke work index (RVSWI) have a key role in predicting and monitoring for post-operative right ventricular dysfunction. Even these advanced hemodynamic parameters that have better prognostic performance have still proven insufficient, in part, at least because the statistical thresholds are derived from population-based thresholds.

### SUMMARY

To transform the predictive capabilities of hemodynamic assessment, an organ, e.g., the heart, should be assessed based on the physiologic tipping points associated with organ performance. By doing so, patient-specific thresholds, rather than statistical, population-based thresholds can be defined which would be more informative for the individual's organ performance assessment and, in turn, in the treatment or therapeutic directives merits in view of that performance.

Patient-specific thresholds for decompensation or recovery are an important and developing area. Using a conductance catheter that can simultaneously measure ventricular volume and pressure, patient-specific pressure-volume (PV) loops can be derived. From this information, the ventriculo-arterial coupling (VAC) ratio can be defined for an individual patient by comparing the elastance of the ventricle (end-systolic elastance, Ees) to the elastance of the downstream circulation (effective arterial elastance, Ea). VAC can be denoted as Ees/Ea and represents the patient-specific relationship of ventricular contractility to the resistive and pulsatile components of afterload of the vasculature. When the ventricle and arterial system are coupled (VAC>1), the ability of the ventricle to contract is matched by the ability of the downstream circulation to accept blood and cardiac ejection, which may lead to better efficiency. When the ventricle and arterial system are uncoupled (VAC<1, particularly when VAC<0.7), the ventricle struggles to eject blood relative to the afterload of the arterial system and efficiency drops.

The PV loop and the VAC provide useful information on both the hemodynamic state and the metabolic condition of a given patient. The hemometabolic state of a patient defines their energy expenditure relative to their energy reserves and tracks clinical outcomes in cardiogenic shock, for example. While measuring PV loops and recording the VAC has increased clinical potential and can provide vital information on the physiological tipping point for a patient, adoption of the PV loop into clinical practice has been slow and it is mostly used as a research tool. Limitations to more widespread use include the technical nature of data acquisition, high cost of the conductance catheters and other specialized equipment and the static nature of the assessment. PV loops are not designed for long-term monitoring and thus when used, they only provide a snapshot in time of the patient's cardiac milieu. Less invasive and less costly techniques such as the single-beat approach can be used but these approaches require computer extrapolations of maximum ventricular pressure and similarly have not been widely adopted.

Here, a system and a device employing a framework, mathematical and physiological in nature, is disclosed. Such a framework delivers highly accurate organ performance through the interplay of hemodynamics and energetics. The framework introduced here defines a true physiological tipping point for the highly accurate assessment of an organ's (e.g., the heart's) performance.

Unlike other methods which have not been widely adopted given the wide-range of technical and financial limitations described above, aspects of the current invention employ commercially available and user-friendly hemodynamic monitoring devices to monitor and determine the hemometabolic state of a patient and their patient-specific tipping point. Although some examples disclosed are directed to specific parameters of the heart, other examples can employ parameters derived from other regions of the body to address cardiovascular performance, or can address various aspects of physiological performance, including in support of healthy individuals.

Examples of methods disclosed allow for a continuous, or near continuous, assessment of the energetic state of a patient, defining, in the case of the heart, the power and efficiency of the left ventricular-aorta unit in isolation, right ventricular-pulmonary artery unit in isolation or the entire cardiovascular system as a whole. With this information about heart function, for example, cardiovascular health can be real-time monitored accurately, and, in some examples, the cardiovascular system may be, in turn, treated in response using particular therapies or devices based on the patient-specific tipping point. In some cases, performance can be modulated in a closed loop system to maximize cardiac performance and longevity.

In one example, the disclosure discloses a cardiac monitoring control system that includes a display device, a detection system to periodically detect one or more parameters at a load, a delivery system coupled to the load, and operable to deliver one or more treatments to the load, and a controller coupled to the display device and the detection system, and includes a processor and a memory storing instructions. When executed, the instructions cause the processor to at least receive signals indicative of the parameters periodically detected at the load transmitted by the detection system, iteratively generate a user-specific ventriculo-arterial coupling (VAC) ratio based on the signals received, control the display device to animate the VAC ratio dynamically, activate the delivery system in response to the VAC ratio, transmit activating instructions, based on the VAC ratio, to the delivery system coupled to the load, and control the delivery system to deliver a user-specific treatment updated dynamically based on the VAC ratio.

In some aspects, the delivery system further comprises at least one of a computerized ambulatory delivery device (CADD) pump to deliver one or more drugs, and a ventricular assist device (VAD).

In some aspects, the user-specific treatment includes at least one of a modeling of the load, and an intervention for the load.

In some aspects, the detection system utilizes at least one pulmonary artery catheter to measure the one or more parameters of the load.

In some aspects, the instructions, when executed, further cause the processor to recognize at least one of a phenotype, a power efficiency, a preserved mechanical reserve, and a clinical event based on the one or more parameters, and to transmit information to the display device based upon at least one of the phenotype, the power efficiency, the preserved mechanical reserve, and the clinical event recognized.

In some aspects, the instructions, when executed, further cause the processor to analyze the signals to generate data indicative of a user-specific myocardial performance (MPS) score.

In some aspects, the instructions, when executed, further cause the processor to generate data indicative of a first alert at the display device when the user-specific MPS score drops below a first threshold, and a second alert at the display device when the user-specific MPS score drops below a second threshold that is below the first threshold.

In another example, the disclosure discloses a method of delivering a user-specific treatment in a cardiac monitoring control system that comprises a display device, a detection system operable to periodically detect a plurality of parameters at a load, a delivery system coupled to the load, and operable to deliver one or more treatments to the load. The method includes transmitting signals indicative of the parameters periodically detected at the load, generating data indicative of a user-specific ventriculo-arterial coupling (VAC) ratio iteratively based on the signals transmitted, animating on the display device the VAC ratio dynamically, activating the delivery system based on the VAC ratio, and transmitting data indicative of the user-specific treatment updated dynamically based on the VAC ratio to the delivery system.

In some aspects, the delivery system further comprises at least one of a computerized ambulatory delivery device (CADD) pump to deliver a drug delivery, and a ventricular assist device (VAD).

In some aspects, the user-specific treatment includes at least one of a modeling of the load, and an intervention for the load.

In some aspects, the method further includes detecting the parameters of the load with a pulmonary artery catheter in the detection system.

In some aspects, the method further includes recognizing at least one of a phenotype, a power efficiency, a preserved mechanical reserve, and a clinical event based on the parameters, and transmitting information to the display device based upon at least one of the phenotype, the power efficiency, the preserved mechanical reserve, and the clinical event recognized.

In some aspects, the method further includes generating data indicative of a user-specific myocardial performance (MPS) score based on the signals.

In another example, the disclosure discloses a non-transitory computer-readable medium storing a plurality of instructions for use with a cardiac monitoring control system for generating a user-specific treatment, the cardiac monitoring control system comprising a display device, a detection system configured to periodically detect a parameter at a load, a delivery system coupled to the load, and operable to deliver one or more treatments to the load, and a controller, coupled to the display device and the detection system. The instructions, when executed, cause the controller to perform the steps of generating signals indicative of the parameters periodically detected at the load detected by the detection system, iteratively animating, at the display device, a user-specific ventriculo-arterial coupling (VAC) ratio based on the signals received, and activating the delivery system in response to the VAC ratio to deliver the user-specific treatment updated dynamically based on the VAC ratio.

In some aspects, the delivery system further comprises at least one of a computerized ambulatory delivery device (CADD) pump to deliver a drug delivery, and a ventricular assist device (VAD).

In some aspects, the user-specific treatment includes at least one of a modeling of the load, and an intervention for the load.

In some aspects, the detection system utilizes at least one pulmonary artery catheter to measure the parameter of the load.

In some aspects, the instructions, when executed, cause the controller to perform the steps of recognizing at least one of a phenotype, a power efficiency, a preserved mechanical reserve, and a clinical event based on the parameter, and transmitting information to the display device based upon at least one of the phenotype, the power efficiency, the preserved mechanical reserve, and the clinical event recognized.

In some aspects, the instructions, when executed, cause the controller to perform the steps of generating data indicative of a user-specific myocardial performance (MPS) score based on the signals.

In some aspects, the instructions, when executed, cause the controller to perform the step of generating data indicative of a first alert at the display device when the user-specific MPS score drops below a first threshold, and a second alert at the display device when the user-specific MPS score drops below a second threshold that is below the first threshold.

Various advantages and features of the present disclosure will become apparent and more clearly understood in view of the detailed description, appended claims, and/or drawings of the present disclosure. In the following description, reference is made to drawings which show by way of illustration various disclosed examples that incorporate various examples of the present disclosure. These examples are described in sufficient detail to enable those skilled in the art to make or use the disclosed examples. Other examples may be utilized and other structural, logical, software, hardware, and electrical changes may be made without departing from the scope of the appended claims. The following description is, therefore, not to be taken in a limited sense.

### BRIEF DESCRIPTION OF THE DRAWINGS

FIG. 1A illustrates relationships of API to Coupling Ratio and Efficiency based on PCWP.

FIG. 1B illustrates relationships of API to Coupling Ratio and Efficiency when PCWP is greater than 20 mmHg.

FIG. 2 illustrates an inverse relationship of API to PE.

FIG. 3 illustrates relationships of CPO to myocardial oxygen consumption (MVO2) for all simulated scenarios and PCWP>20.

FIG. 4 illustrates relationships of predicted PE to MPS and mechanical efficiency.

FIG. 5 illustrates a modeling of the Starling Curve using Michaelis-Menten kinetics.

FIG. 6 illustrates a modeling of patient specific Starling curves using Km=2/API.

FIG. 7 illustrates correlations of MPS and Power Efficiency Surrogate with metabolic efficiency.

FIG. 8 illustrates correlations of MPS and Power Efficiency Surrogate with mechanical efficiency.

FIG. 9 illustrates relationships of MPS to Power Efficiency, mechanical efficiency (SW/PVA) and CPO.

FIG. 10 illustrates a load-independent MPS and Power Efficiency predict mechanical efficiency.

FIG. 11 illustrates a balance of power and potential energy by MPS.

FIG. 11A illustrates a first myocardial performance map.

FIG. 11B illustrates a second myocardial performance map.

FIG. 11C illustrates heart failure predictions based on API and CPO.

FIG. 11D illustrates exemplary Risk Stratifications.

FIG. 12 illustrates a Sine transformation of MPS.

FIG. 13 illustrates an interpretation of the Starling curve in the context of power output, efficiency and MPS.

FIG. 14A generally illustrates a comparison of the hemodynamic profile to the myocardial performance profile.

FIG. 14B generally illustrates a relationship between MPS, power, efficiency, and potential energy.

FIG. 14C illustrates a mechanical efficiency.

FIG. 15 illustrates relationships of API and CPO to coupling ratios.

FIG. 16 illustrates static and dynamic assessments of the prognostic role of API and/or CPO to predict 1 year survival free from LVAD or transplant.

FIG. 17 illustrates a risk stratification based on initial MPS and final MPS after milrinone infusion.

FIG. 18 illustrates Mechanical Efficiency predicted from load-independent MPS.

FIG. 19 illustrates an exemplary diagram showing a monitoring system.

FIG. 20 illustrates an exemplary example of a general monitoring system.

FIG. 21 illustrates different exemplary applications of a general monitoring system.

The figures are not necessarily to scale. Various dimensions may be exaggerated for illustrative clarity. Where appropriate, similar or identical reference numerals are used to refer to similar or identical components.

### DETAILED DESCRIPTION

Implementations of the present disclosure represent a technical improvement in the art of medical technology. Specifically, the implementations illustrated address the technical problem of organ failure risk, e.g., cardiac failure risk as experienced by patients. As described above, the hemometabolic state of a patient defines energy expenditure relative to energy stores or reserves. When the transfer of energy from the ventricle to the blood elements as they are ejected into the downstream circulation is efficient, energy expenditure is minimized, and finite energy reserves are preserved. The efficiency of this energy transfer may be defined as the mechanical efficiency of the heart.

Aspects described herein act as a practical implementation of an assessment of a patient's organ failure risk by using a patient's hemometabolic state to provide one or more specific treatments for the patient, such as providing specific, customizable drug delivery for the patient, indicating that the patient is suitable for an intervention, and/or monitoring a patient after an intervention. In some examples, the hemometabolic state may be analyzed in order to categorize the patient based on risk stratifications which may be used by a provider to recommend a treatment and/or which may be used to customize drug delivery to the patient.

In some aspects, the system described herein uses a unique algorithm or executable code, which is implemented in computing devices, including, but not limited to desktop computers, laptops, mobile or smart electronic devices such as mobile phones, smart wrist watches, wearable devices, and tablets, plug-and-play hardware, computer components, computer boards, smart cards, chip cards, integrated circuit cards, microchips, display devices, implantable devices, and bedside patient monitors, and computing devices processes, including, but not limited to mobile device smart device apps, embedded programs, non-transitory memory devices, graphical user-interfaces (GUI), server or network software, and installable software, to analyze and/or determine the hemometabolic state of a patient and/or the mechanical efficiency of an organ. The specific and practical application of such an algorithm or executable code may be used to determine a customized drug dosage for a patient and/or to more accurately assess whether a patient would perform well with an organ transplant. In some examples, the algorithm or executable code may be implemented in one or more computing devices and used to more accurately monitor and assess a patient after an organ transplant. In some examples, the algorithm or executable code may be used to analyze large amounts of data collected from a patient (continuously, periodically, in real-time, etc.) and provide an assessment to a user. In some examples, the assessment may be used to determine dosage, drug information, etc., that should be provided to a user. The assessment may include graphs, pictures, icons, or other visual information so that a user may efficiently determine the status of a patient and/or whether intervention is needed. In some examples, the assessment may be displayed in an efficient format to aid a practitioner in recommending a course of treatment for a patient. In some examples, the assessment may directly or indirectly cause a graphical user interface or display to modify a displayed content to accommodate any additional or dynamically changing information due to its size limitation. In some examples, the algorithm implemented in the computing devices may be used to continue monitoring a patient in order to update dosage or drug delivery information and/or to modify the assessment regarding the course of treatment, for example, during different periods of treatments for the patient, thus providing an iterative and/or continuous assessment of information and/or signals obtained from the patient. Such analysis and assessments are specific, practical, and provide a technological improvement to medical technology.

### Pressure-Volume Loop Model of Cardiac Input and Output

Mechanical efficiency in cardiac assessment represents the proportion of the heart's total energy expenditure that directly is responsible for ejection of blood during systole. The work of ejection is defined as the stroke work (SW), and it can be represented on the PV curve as the area contained in the PV loop itself. The total energy expenditure of the heart can be represented by the area contained by the boundary of the end-systolic pressure volume relationship (ESPVR) and end-diastolic pressure volume relationship (EDPVR) and is referred to as the pressure volume area (PVA). As the heart becomes more uncoupled, the proportion of total energy expenditure (PVA) that is directly applied to systolic ejection (SW) decreases and more energy is needed to prepare the heart for ejection (potential energy, PE) which includes the energy needed for calcium handling and myofilament positioning, leading to a reduction in mechanical efficiency.

The stroke work (SW) can be approximated as:

SW=MAP×SV,

where MAP is mean arterial pressure, and SV is stroke volume. The Potential Energy (PE) can be approximated as:

$$PE = \frac{MAP \times ESV}{2} ,$$

where ESV is end-systolic volume. Pressure volume area (PVA) is the sum of SW and PE:

PVA=SW+PE

Thus, cardiac or mechanical efficiency (Eff) can be approximated as:

$$Eff = \frac{SW}{PVA} , or \\ Eff = \frac{SW}{SW + PE} .$$

The ratio of useful energy (SW) to non-useful potential energy (PE) is thus:

$$\frac{SW}{PE} = \frac{MAP \times SV}{0.5 MAP \times ESV} , or \\ \frac{SW}{PE} = \frac{2 SV}{ESV} .$$

From the above relationships, efficiency will decrease when the rate of change of PE outpaces the rate of change of SW. PE equals SW when ESV is twice the value of SV (stroke volume). In some cases, loading conditions refer to the volume, pressure, or strain on the ventricular walls. As ventricular volume increases, pressure increases as does wall strain. At loading conditions, beyond this point, mechanical efficiency (Eff) decreases with increasing loading conditions as potential energy increases out of proportion to stroke work. At the cellular level, this represents a point where the enthalpy of cross-bridging of the actin-myosin unit exceeds useful mechanical work.

From a physiological perspective, mechanical efficiency (Eff) can be thought of as the ratio of output energy from the heart divided by input energy to the heart. In the above mathematical relationship, SW also represents the output of the heart. Output of the heart can also be represented as stroke volume, cardiac output, and cardiac power. The input energy to the heart in the above example is represented as SW+PE but often more clinically relevant surrogates which track with input energy are substituted for SW+PE such as end-diastolic volume (EDV), end diastolic pressure (EDP), or its surrogate pulmonary capillary wedge pressure (PCWP). When output is plotted against input, the Starling relationship of the heart is obtained. Mechanical efficiency (Eff) is maximal when output far outpaces input and approaches zero as input far outpaces output. From the above relationship, the heart is 50% efficient when the input of the heart is twice the output of the heart.

Clinically, the balance of output and input may be described in terms of ventriculo-arterial coupling (VAC). The ventricle and downstream circulation are coupled when the elastance of the ventricle (Ees, end-systolic elastance) is matched by the elastance of the circulation (Ea, effective arterial elastance). The ratio of Ees/Ea represents the VAC ratio. Ees is represented on the PV as the slope of the end-systolic pressure volume relationship (ESPVR) while Ea is represented by a slope of the line that intercepts ESP and EDV. ESP can be approximated by 0.9 SBP (spontaneous bacterial peritonitis) or by mean arterial pressure (MAP).

End-systolic elastance can be approximated as:

$$Ees = \frac{ESP}{ESV} ,$$

where ESP is the end-systolic pressure. Arterial elastance can be approximated as:

$$Ea = \frac{ESP}{SV} .$$

The coupling ratio can therefore be approximated as:

$$\frac{Ees}{Ea} = \frac{SV}{ESV} .$$

Given that efficiency precipitously declines when the rate of change of PE>SW, Ees/Ea tracks with mechanical efficiency in a predictable fashion when the system is sufficiently loaded.

### Starling Curve Model of Cardiac Input and Output

The complicated relationship between mechanical efficiency (Eff), the coupling ratio, input of the heart and output of the heart can further be understood by employing the Starling model of the heart. In the most traditional form of the Starling curve, the input is often denoted as ventricular pressure (RAP, LAP or PCWP) or volume (RVEDV, LVEDV) at end-diastole. The traditional representation of output of the heart is either SV or CO, however it can also be represented by ventricular stroke work (SW) or cardiac power output (CPO).

The slope of the Starling curve therefore represents mechanical efficiency (Eff) as it denotes useful output relative to input. At lower ventricular volumes and pressures, the rate of change of output relative to input is high (steep slope on Starling curve, high efficiency) while at larger ventricular volumes, the rate of change of output begins to slow down relative to the rate of change of input and efficiency decreases. At the point that the rate of change of output matches the rate of change of the input, the system is balanced. As described above, this occurs when ESV is twice SV. Incremental loading beyond this point only nominally affects SV (plateau in Starling curve) and thus efficiency as well as the ratio of SWIPE and the coupling ratio all track with ventricular loading conditions beyond this inflection point.

### Aortic Pulsatility Index and Pulmonary Artery Pulsatility Index

If alternatively, the output is represented by pulse pressure and the input by right atrial pressure (RAP) (for the right ventricle, RV) or PCWP or LAP (for the left ventricle, LV). It can be better understood the clinical relevance of the advanced hemodynamic parameters, pulmonary artery pulsatility index (PAPI, (PAs-Pad)/RAP) and aortic pulsatility index (API, (SBP-DBP)/PCWP). API and PAPI are important hemodynamic parameters utilized in prognostication and optimization of the right and left ventricles, respectively. They reflect the pulsatile load of ejection on the vasculature for a given ventricular preload and thus mechanistically they reflect the ventriculo-arterial unit. The difference between systolic and diastolic blood pressure or pulse pressure (PP) is on the numerator for both PAPI and API. Pulse pressure is determined by SV and Ea:

PP=SV×Ea.

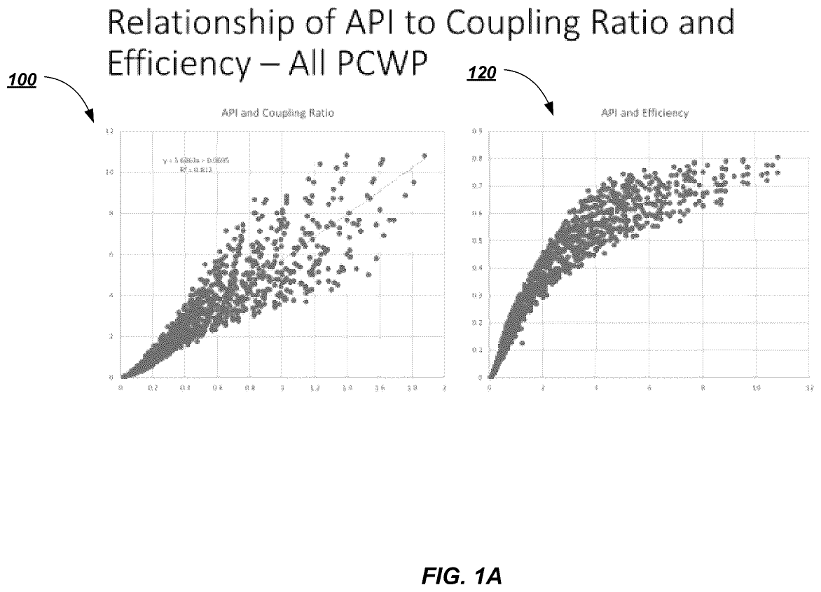

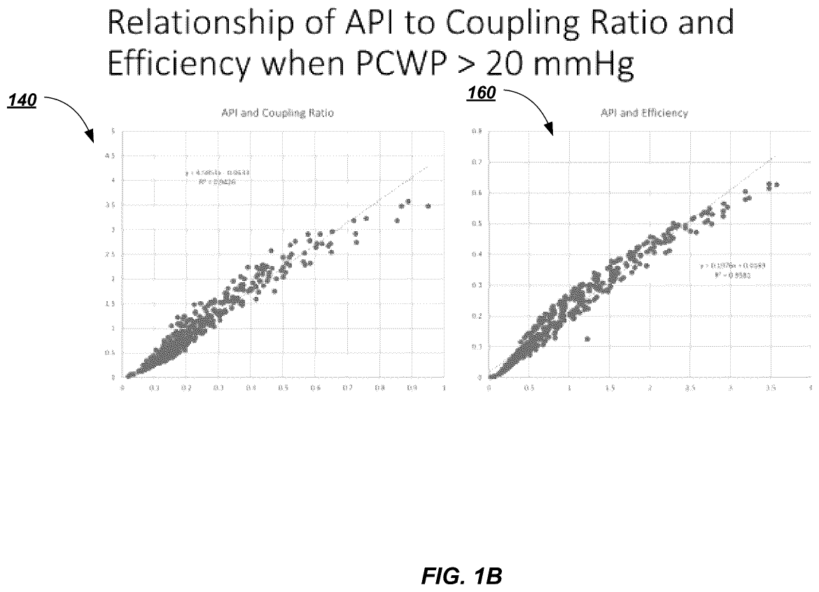

For a given Ea, API is proportional to SVIPCWP or SVILAP and PAPI is proportional to SV/RAP. API and PAPI therefore reflect the slope of the Starling input/output curves for a given Ea. The Starling curve for an individual patient is established by the contractile state of the ventricle (Ees) and the resistive and pulsatile afterload of the downstream vasculature (Ea). Thus, API and PAPI are also reflective of the coupling ratio of the ventricle (e.g., VAC). The higher the API or PAPI, the more efficient the transfer of blood and energy from ventricle to vasculature. These relationships become more linear as loading conditions increase. FIG. 1A illustrates a relationship of API to Coupling Ratio 100 and Efficiency 120 based on PCWP. FIG. 1B illustrates a relationship of API to Coupling Ratio 140 and Efficiency 160 when PCWP is greater than 20 mmHg.

### Potential Energy Determinants

As discussed above, efficiency decreases when the rate of change of PE is greater than the rate of change of SW. At the cellular level, as ventricular strain increases, the actin-myosin units reach their limits of stretch and further stretch leads to inefficient cross bridging with heat production outpacing useful mechanical work. At the macro level, as vascular stress increases, the arterial walls reach their limits of stretch and any further transfer of energy from the ventricle to the arterial system is transmitted as kinetic energy as the elastic potential energy of the aorta and its branches becomes saturated. When this occurs, the velocity of pulse wave propagation down the arterial system increases. Both antegrade and retrograde wave propagation is enhanced with the latter opposing net antegrade flow and cardiac ejection when the wave reflections propagate back to the ascending aorta during systole. The net effect from incremental loading is an increase of PE from both mechanisms.

The actin-myosin relationship as well as the limits of vascular stretch may be modeled using spring theory to better understand the determinants of PE. The relationship of potential energy to stress and strain is well defined:

$$PE = \frac{1}{2} stress \times strain .$$

Young's modulus (E) defines the tensile stiffness of a material and represents the ratio of stress (σ) to axial strain (ε) of a material. In vascular biology, Young's modulus can define the tensile properties of the vasculature and is referred to as the pressure/strain elastic modulus, E=K (Ps−Pd)/strain, where K is a constant.

Given that E=stress/strain, the potential energy or strain per unit volume can be calculated from E as follows:

$$PE = 1 / 2 {( stress )}^{2} / E .$$

Thus, E is inversely proportional to potential energy.

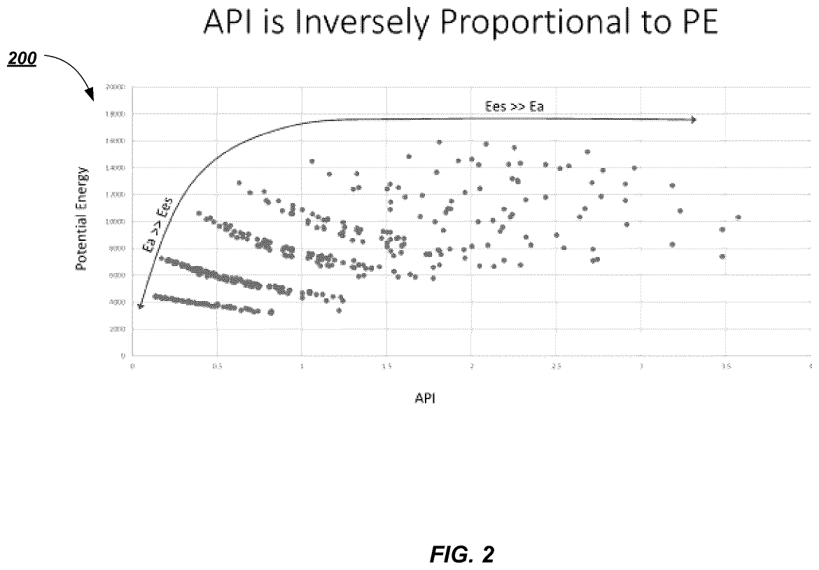

Given that API and PAPI represent stress/strain of the ventriculo-arterial unit for the left and right ventricle, respectively it reasons that PE is also inversely proportional to API and PAPI. The relationship becomes more defined as the ventricle becomes progressively more uncoupled when the influence of Ea on pulse pressure becomes more dominant as shown in FIG. 2. Specifically, FIG. 2 illustrates an inverse relationship 200 of API to PE. For a given Ees, there is a linear relationship of API to PE. The relationship is best defined when Ea>Ees.

Summarizing the above, the following relationship exists between API/PAPI, cardiac efficiency the coupling ratio and potential energy:

API∝Eff_{LV}∝LV Ees/Ea ∝1/PE_{LV},

PAPI∝Eff_{RV}∝RV Ees/Ea∝1/PE_{RV}.

### Cardiac Power Output and Cardiac Power Efficiency

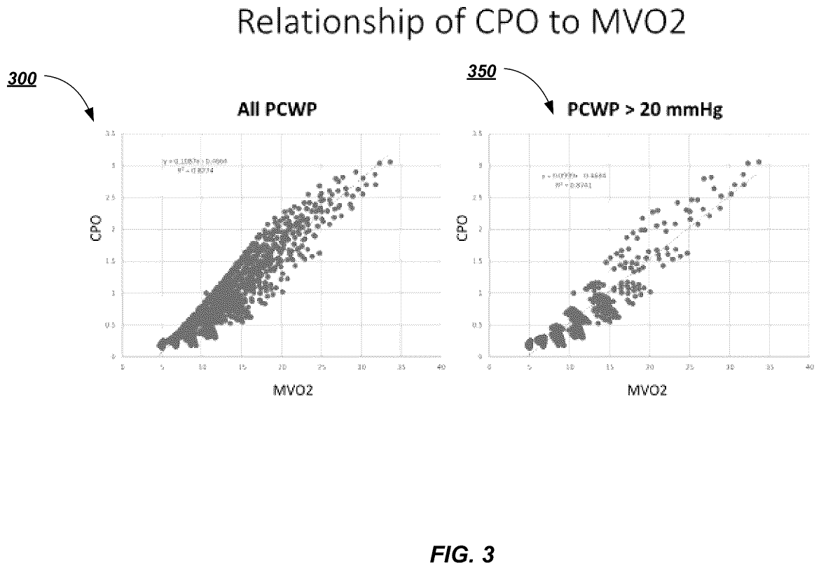

Cardiac power output represents the energy output per unit time of the cardiovascular system. From the PV loops, CPO can be calculated by multiplying ventricular stroke work (SW) by heart rate (HR), or SW×HR. CPO can also be estimated as MAP×CO/451. SW and CPO represent the mechanical work and power of the heart and are directly proportional to total ventricular energy expenditure and myocardial oxygen consumption (MVO2) as shown in FIG. 3.

Specifically, FIG. 3 illustrates relationships of CPO to myocardial oxygen consumption (MVO2) for all simulated scenarios 300 and PCWP>20, 350. CPO is a highly prognostic variable in patients in cardiogenic shock after an acute myocardial infarction (AMI). A value of less than 0.6 W (which roughly translates to a MAP of 65 mmHg and CI of 2.2 L/min/m² for the average sized individual) is often targeted as the minimal acceptable power output when managing patients with shock. While CPO remains highly prognostic in AMI cardiogenic shock (CS-AMI), its prognostic potential in cardiogenic shock secondary to heart failure (CS-HF) has been less consistent.

After the onset of heart failure, activation of the renin-angiotensin-aldosterone axis leads to retention of salt and water. Vascular tone is also increased to maximize perfusion to vital organs. Consequently, the PV loop for a patient with CS-HF can be rightward shifted. If the patient has adequate myocardial reserve, stroke work and thus CPO, can be maintained for some time but at the expense of cardiac efficiency and increased intracardiac filling pressures. To better conceptualize the prognostic role of CPO, it is necessary to discuss CPO in the context of cardiac efficiency.

As discussed above, mechanical efficiency is the ratio of energy output to energy input. At the cellular level, the energy input of the myofilaments is the sum of the activation heat (Q_{A}), crossbridge heat (Q_{XB}) and external work (W). Q_{A} is a constant and independent of loading conditions whereas W and Q_{XB} will increase with increasing preload or afterload. Thus, efficiency can be represented as:

$$Eff = \frac{W}{QA + W + QXB} .$$

As preload increases, Q_{A} becomes small relative to W and Q_{XB} and the relationship can be simplified as:

$$Eff = \frac{W}{W + QEB} .$$

When the rate of change of crossbridge heat is greater than the rate of change of work, efficiency will decrease. Said another way, when the rate of change of energy input exceeds the rate of change of energy output, efficiency will drop.

At the macro level, as described above, energy input is the sum of stroke work and potential energy whereas energy output is equal to stroke work. Since CPO is SW×HR, the power efficiency (P_{Eff}) may be defined as:

$${P}_{Eff} = \frac{CPO}{CPO + PE} .$$

When the rate of change of potential energy is greater than the rate of change of external power, power efficiency will decrease.

From the above derivations, PE is inversely proportional to API and PAPI times a constant (K_{LV} (left ventricle constant) and K_{RV} (right ventricle constant), further detailed below), particularly when the ventricle is uncoupled. P_{Eff} for the left ventricle may be defined as follows:

$${P}_{{Eff}_{LV}} = \frac{CPO}{CPO + \frac{{K}_{LV}}{API}}$$

P_{Eff} for the right ventricle may be defined as follows:

$${P}_{{Eff}_{RV}} = \frac{CPO ( RV )}{CPO ( RV ) + \frac{{K}_{RV}}{PAPI}}$$

API is a dimensionless parameter. To calibrate the system, K may be defined for both the right ventricle and left ventricle. If the tipping point for the system is defined as the point where the rate of change of useful work equals the rate of change of potential energy, P_{Eff} at this point would be 50%. CPO is a derived value with the constant 451 defined to normalize CPO to 1 W for the typical patient with blood pressure 120/80 mmHg, MAP 93.3 mmHg, RA pressure of 3 mmHg and CO of 5 L/min.

Under these same idealized conditions, using a PCWP value of 10 mmHg, API will be equal to 4 [(120/80 mmHg)/10 mmHg]. The healthy human heart has an Ees/Ea of about 2. Said another way, the healthy human heart has a mechanical efficiency of 66.67% or an energy output which is twice potential energy.

The left ventricular power efficiency may be calibrated using the normalized values of CPO and API. K_{LV} may be defined as follows:

$${P}_{{Eff}_{LV}} = \frac{CPO}{CPO + PE} = 66.67 \% or 2 / 3 ,$$

$${P}_{{Eff}_{LV}} = \frac{CPO}{CPO + \frac{KLV}{API}} = 66.67 \% or 2 / 3 , and$$

$${P}_{{Eff}_{LV}} = \frac{1}{1 + \frac{KLV}{4}} = 2 / 3.$$

### Solving for K_{LV}:

K_{LV}=2.

P_{Eff} for the left ventricle may be estimated as follows:

$${P}_{{Eff}_{LV}} = \frac{CPO}{CPO + \frac{2}{API}} .$$

### And at a P_{Eff} of 50%, 2/API=CPO.

To calibrate the power efficiency of the right ventricle, the typical pulmonary artery (PA) pressures may be defined as 25/10 mmHg with a mean PA pressure of 15 mmHg. Using an RA pressure of 3 mmHg and CO of 5 L/min, the reference standard for CPO_{RV} is:

$${CPO}_{RV} = \frac{( mPA - RA ) \times CO}{451} ,$$

$${CPO}_{RV} = \frac{( 15 mmHg - 3 mmHg ) \times 5 L / min}{451} , and$$

$${CPO}_{RV} = 0.133 or \sim 2 / 15.$$

And the reference standard for PAPI is:

$$PAPI = \frac{PAs - PAd}{RA} ,$$

$$PAPI = \frac{25 mmHg - 10 mmHg}{3 mmHg} , and$$

$$PAPI = 5.$$

Unlike the left ventricle which, when healthy, has Ees/Ea of about two or P_{Eff-LV} of 66.67%, the healthy right ventricle operates at a P_{Eff RV} closer to 50%.

The right ventricular power efficiency may be calibrated and K_{RV} defined as follows:

$${P}_{{Eff}_{RV}} = \frac{CPO ( RV )}{CPO ( RV ) + \frac{KRV}{PAPI}} = 50 \% or 1 / 2 ,$$

$${P}_{{Eff}_{RV}} = \frac{\frac{2}{15}}{\frac{2}{15} + \frac{KRV}{5}} ,$$

### Solving for K_{RV}:

$${K}_{RV} = \frac{2}{3} .$$

P_{Eff} may be defined for the right ventricle as follows:

$${P}_{{Eff}_{RV}} = \frac{{CPO}_{RV}}{{CPO}_{RV} + \frac{2}{3 PAPI}} .$$

At a P_{Eff} of 50%, 2/(3PAPI)=CPO_{RV}.

The above equations for K_{LV} and K_{RV} assume that the relationship of useful power to PE and 2/API or 2/(3PAPI) are linear for different loading conditions and can be approximated by the power/PE relationship when P_{E}gf=50%. While this assumption holds for many conditions and can be used in most clinical situations, the more accurate representation of PE is:

$$PE = \frac{{2}^{\frac{1}{MPS}}}{API} AND PE = \frac{{2}^{\frac{1}{MPS}}}{3 PAPI}$$

Below further details a definition of myocardial performance score (MPS). When MPS is one, the equation simplifies to 2/API and 2/3(PAPI).

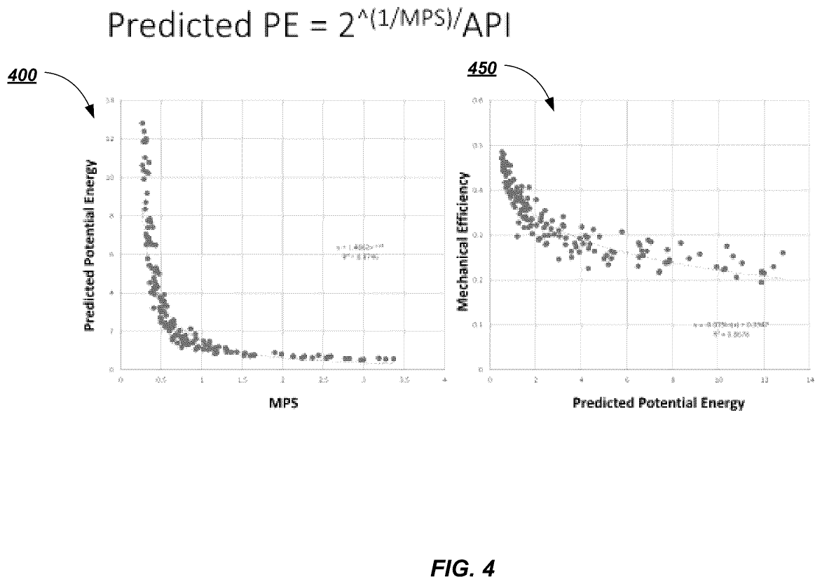

As shown in FIG. 4, plotting the predicted PE vs the MPS or mechanical efficiency provides the relationships. Specifically, FIG. 4 illustrates relationships of predicted PE to MPS 400 and mechanical efficiency 450. PE exponentially increases when the MPS<0.5. In this simulation, MPS scores of <0.25 were excluded given the exponential relationship of 1/MPS to PE leading to a large splay in the data.

### Modeling Patient-Specific Starling Curves

From Starling's law of the heart, for a given contractility and afterload, energy output of the heart increases with preload until the limits of actin-myosin stretch are reached and output plateaus. At the cellular level, overstretching of the actin-myosin unit leads to inefficient cross-bridging, an elevation in potential energy relative to stroke work and cardiac efficiency decreases. Thus, the limit of power output of the heart is determined by the rate of change of potential energy. A highly efficient system will have a reduced rate of change of potential energy than a less efficient system leading to an enhanced rate of change of stroke work and cardiac power output.

The rate of change of power output may be modeled using Michaelis-Menten Kinetics. The reaction of converting chemical energy (metabolic fuel) into mechanical energy (actin-myosin cross bridging and power stroke) is dependent on the availability of metabolic substrates and the efficiency actin-myosin cross-bridging. The efficiency is, in turn, dependent on the initial length of the myofilaments which is itself dependent on the stress and strain in the ventricle. Near the limits of stretch, potential energy increases rapidly and instantaneous crossbridge efficiency approaches zero. Thus, the Starling curve may be modeled as a percentage of maximum power output (when efficiency approaches 0%) which will correspond with maximum power output for the patient or CPO_{Max}. The relationship follows Michaelis-Menten Kinetics as follows:

$$Vo = \frac{{CPO}_{Max}}{Km + Ventricular load} ,$$

where V₀ is the reaction rate, ventricular load (V_{load}) is a loading condition of the ventricle and Km is the Michaelis-Menten constant.

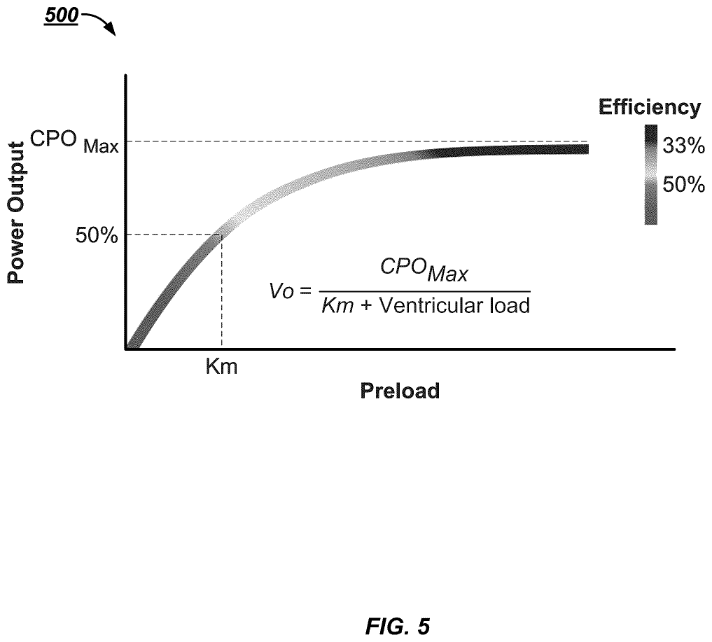

As shown in FIG. 5, using Michaelis-Menten kinetics, Km represents the stress/strain relationship when power output is 50% maximum efficient, which illustrates a modeling of the Starling Curve using Michaelis-Menten Kinetics 500.

As described above, API and PAPI are inversely proportional to PE and is indicative of the efficiency of energy transfer and the stress/strain relationship. The previously calibrated system balances power output and potential energy and the point of 50% mechanical efficiency using 2/API and 2/(3PAPI) for the left and right ventricles, respectively. Thus, the patient-specific Starling curve can be defined for a given contractility and afterload state using 2/API for the LV and 2/(3PAPI) for the RV as the Km for the curve.

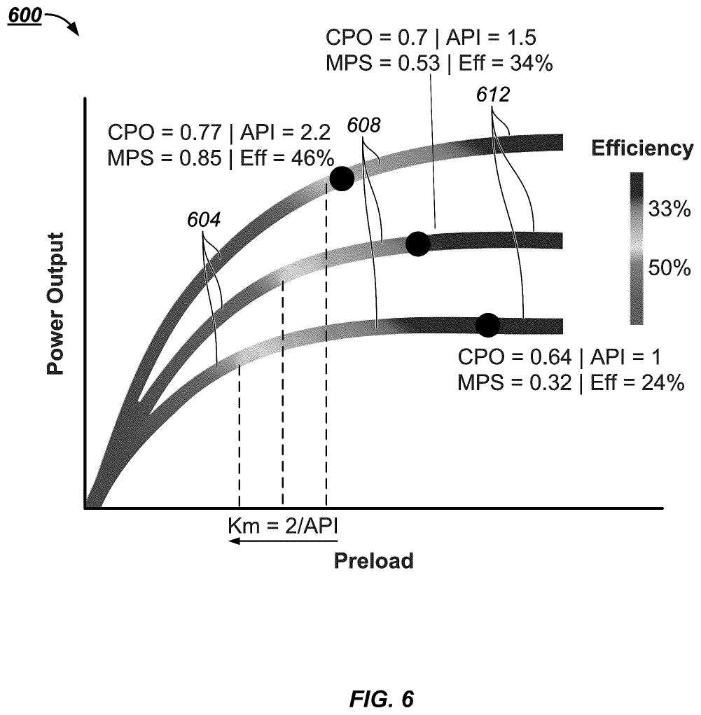

As shown in FIG. 6, as efficiency increases, Km will decrease, and the Starling curve will shift up and to the left. Specifically, FIG. 6 illustrates a modeling 600 of a patient specific Starling curve using Km=2/API. The zone of energy recovery is denoted in green 604 and correspond to an MPS>1 (>50% power efficiency). Zones of borderline (yellow 608, MPS 0-1) and rapid (red 612, MPS<0.5, power efficiency<33.3%) energy depletion are also noted.

### Energetic Phenotyping: Myocardial Performance Profile and the Myocardial Performance Score

Taken together API/PAPI and CPO supply additive information to help define the hemometabolic state of a given patient which can be described in terms of the myocardial performance profile. CPO stands for the energy expenditure of the heart and API/PAPIrepresents the efficiency of energy handling. A high performing heart, like a high performing combustion or electric engine, can generate maximum power with high efficiency. To maintain adequate tissue perfusion, a minimal CPO value of about 0.6 W is needed under most conditions (to maintain MAP>65 mmHg and CI>2.2 L/min/m²). Thus, the ideal myocardial performance profile is one that maximizes efficiency and ventricular coupling (high=API or PAPI) while at the same time can maintain a CPO>0.6 W.

The power-efficiency relationship or myocardial performance can be represented by combining API and CPO into a singular variable called the myocardial performance score (MPS). Given the inverse relationship of API and PAPI to PE, the product of CPO and API or PAPI stands for the ratio of useful external work to potential energy. MPS of 1 may be defined as the point where power output and potential energy are equal.

As discussed above, the calibrated API where PE=CPO for the LV is 2/API and the calibrated PAPI where PE=CPO for the RV is 2/(3PAPI).

Thus, the myocardial performance score for the LV is:

$${MPS}_{LV} = \frac{CPO \times API}{2} .$$

The myocardial performance score for the RV is:

$${MPS}_{RV} = \frac{3 CPO \times PAPI}{2} .$$

Power efficiency (P_{Eff}) is the ratio of power output (Pₒᵤₜ) to power input (Pᵢₙ). Pₒᵤₜ is represented by CPO and Pᵢₙ for the LV can be represented by CPO+2/API given the inverse relationship of API to potential energy:

$${P}_{Eff} Surrogate = \frac{Pout}{Pin} , and$$

$${P}_{Eff} {Surrogate}_{LV} = \frac{CPO}{CPO + \frac{2}{API}} .$$

And P_{Eff} for the RV can be represented as:

$${P}_{Eff} {Surrogate}_{RV} = \frac{{CPO}_{RV}}{{CPO}_{RV} + \frac{2}{3 PAPI}}$$

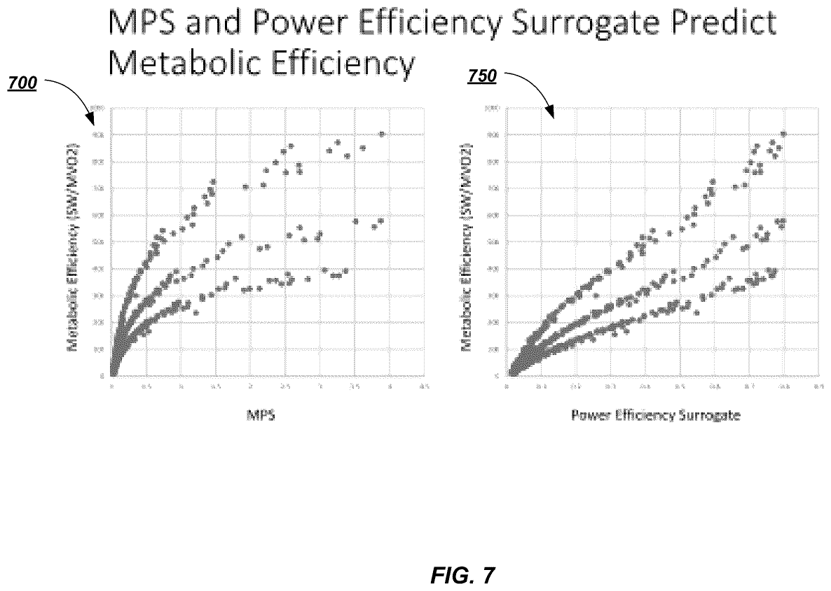

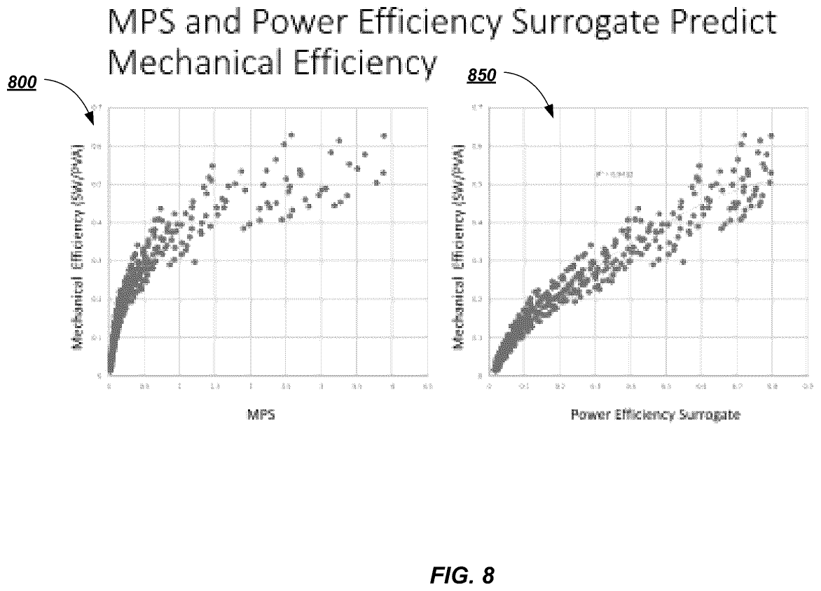

MPS and P_{Eff} Surrogate correlate strongly with metabolic efficiency as shown in FIG. 7 and mechanical efficiency as shown in FIG. 8. Specifically, FIG. 7 illustrates correlations of MPS 700 and Power Efficiency Surrogate with metabolic efficiency 750, and FIG. 8 illustrates correlations of MPS 800 and Power Efficiency Surrogate with mechanical efficiency 850.

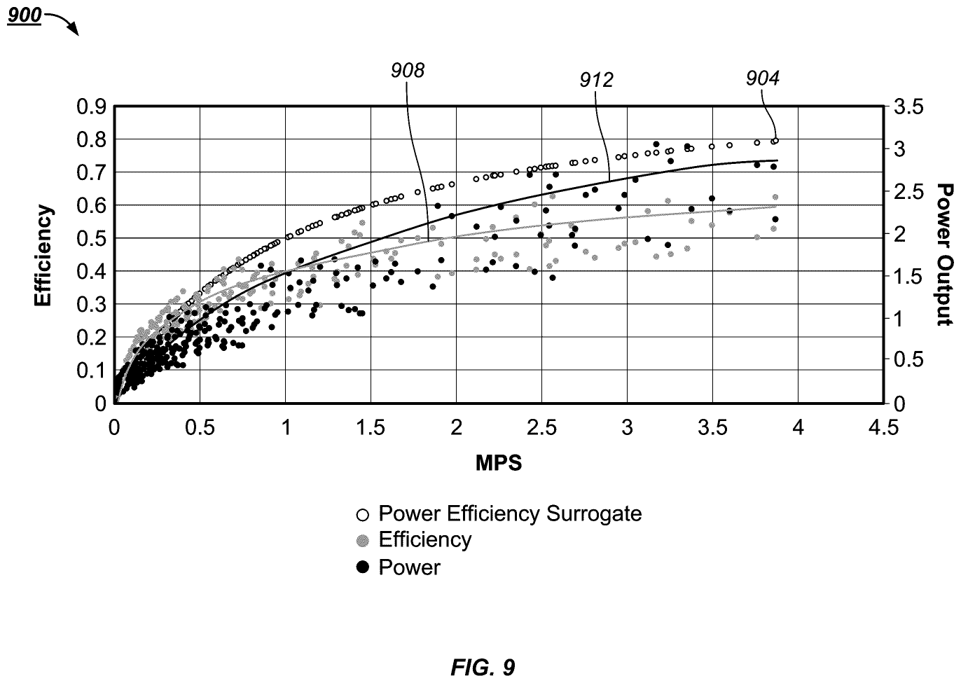

When P_{Eff} efficiency (SW/PVA) and power (CPO) are plotted against MPS, relationship as shown in FIG. 9 results. Specifically, FIG. 9 illustrates relationships 900 of MPS to Power Efficiency 904, mechanical efficiency (SW/PVA) 908, and CPO 912. Specifically, FIG. 9 shows that there is a precipitous drop in efficiency (SW/PVA) at an MPS score of <0.5. The mechanical efficiency (SW/PVA) 908 rapidly drops or decelerates at a score of 0.5. This represents the tipping point for the patient.

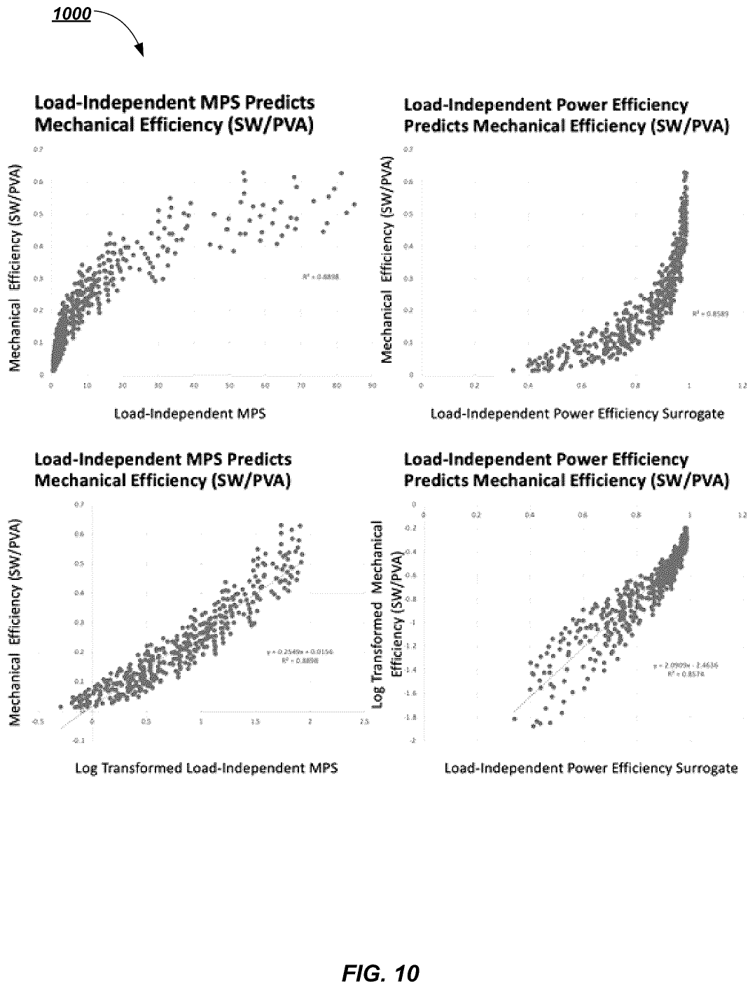

FIG. 10 shows that relationships 1000 between MPS and P_{Eff} with efficiency holds true even when the loading conditions are not known. Specifically, FIG. 10 illustrates a load independent MPS, and power efficiency predict mechanical efficiency. As discussed above with respect to FIG. 9, an MPS of 0.5 represents the tipping point where mechanical efficiency drops precipitously. A potential restriction or limitation of the MPS score is that it requires knowledge of the filling pressures which means it involves an invasive procedure (right heart catheterization). FIG. 10 shows that a strong estimate of both MPS and mechanical efficiency from a load-independent surrogate (LI-MPS and LI-Peff) may be obtained. This may mean that devices such as a wristwatch or the implantable PA sensor can be used with high accuracy and loading conditions are not a pre-requisite.

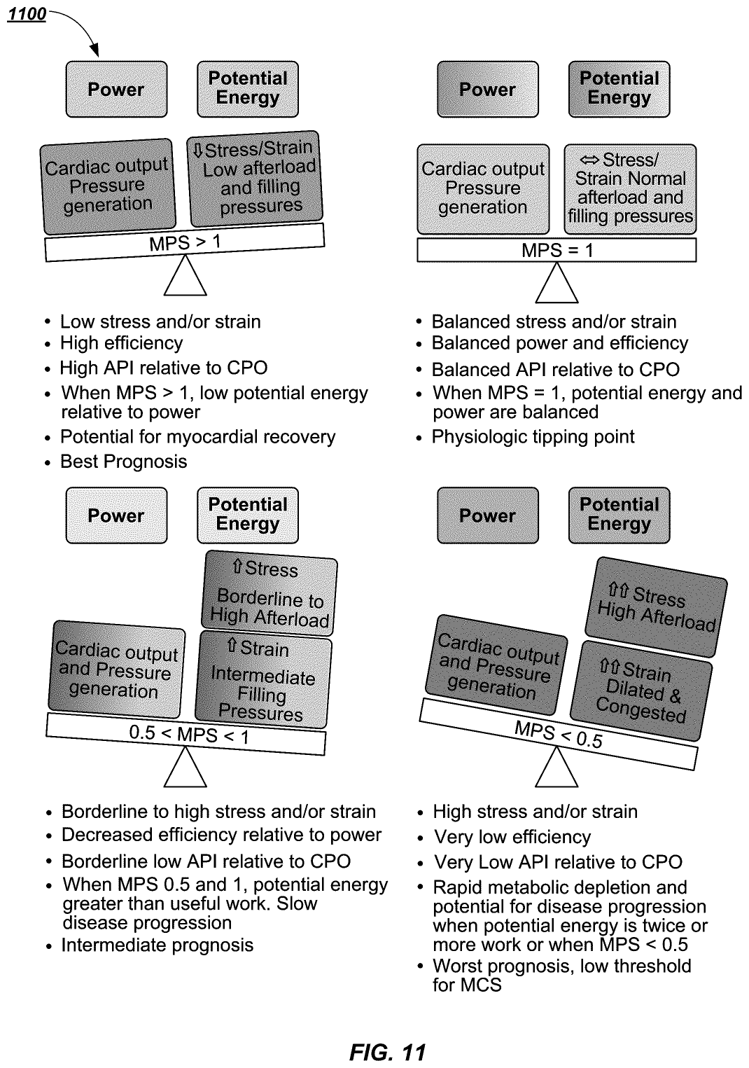

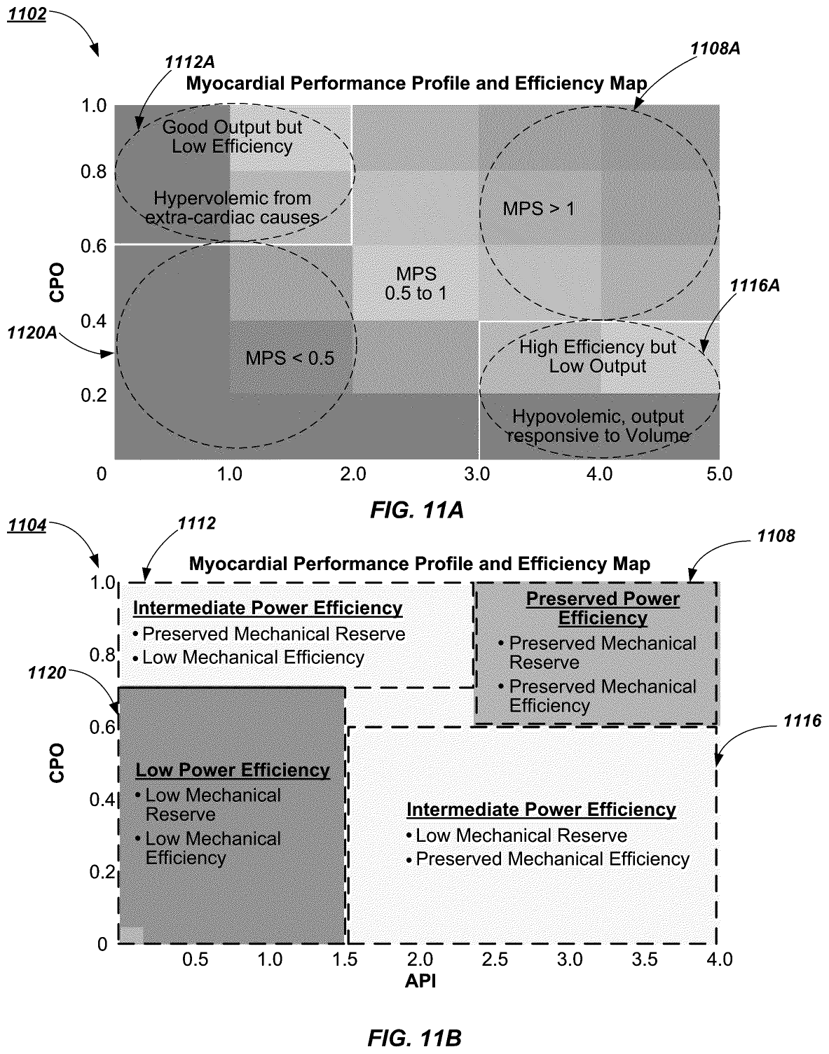

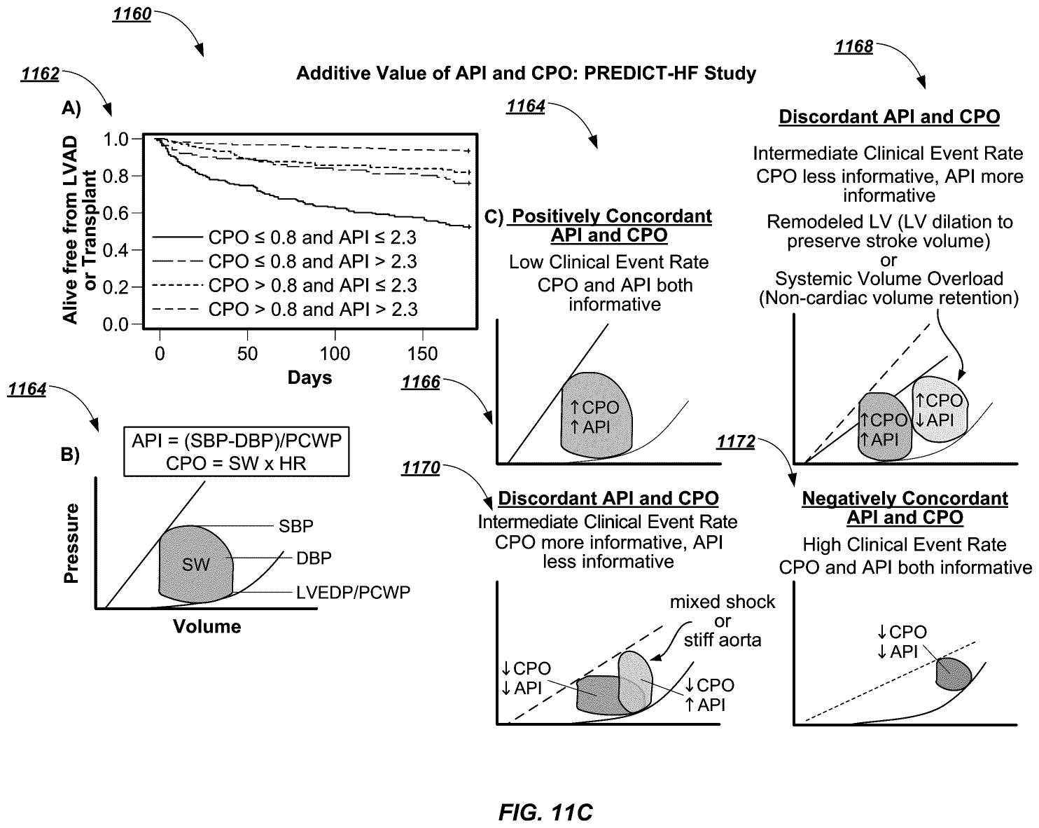

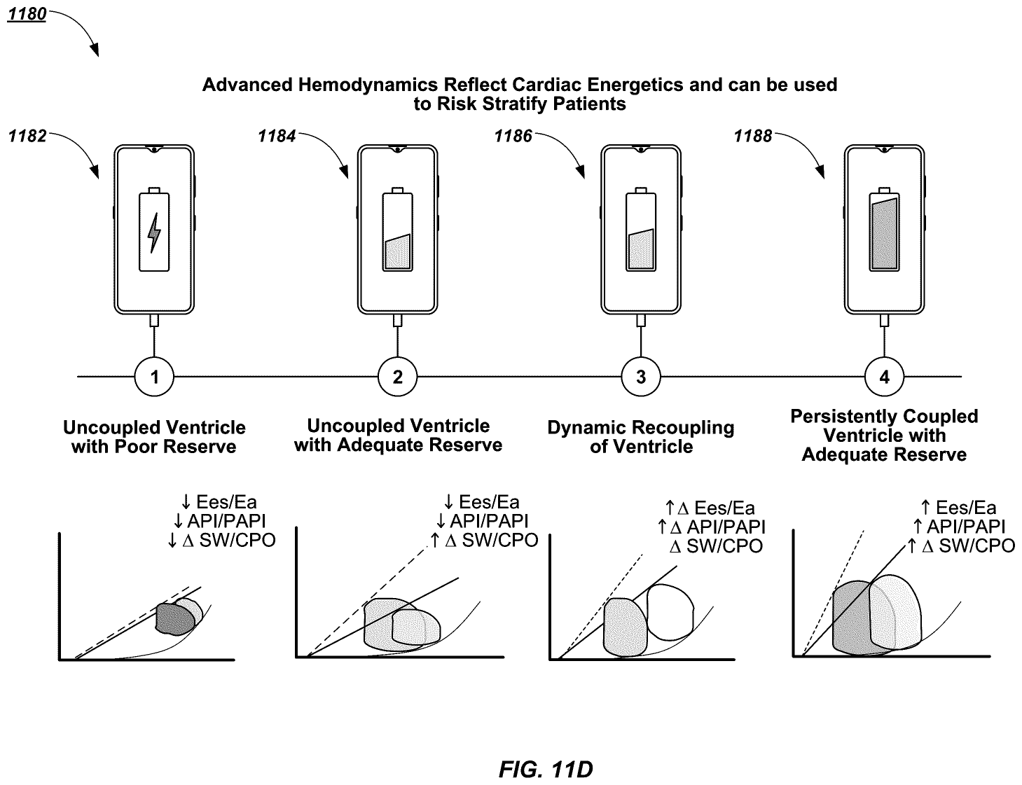

As shown in FIGS. 9 and 11, at MPS of 1, power efficiency is 50% and is the point of balance between power and efficiency. At MPS of 2, the efficiency relative to power is maximum and at MPS<0.5, there is a rapid decline in efficiency. FIG. 11 also illustrates a balance of power and potential energy by MPS 1100. This goes back to how the MPS was derived. By definition, an MPS of 1 represents the point where power output and potential energy are equal. An MPS of 2 is the point where power is twice potential energy and an MPS of 0.5 is when potential energy is twice the power output. The Peff at an MPS of 0.5 is thus 33.33%, the Peff at an MPS of 1 is 50% and the Peff at an MPS of 2 is 66.67%.

FIGS. 11A and 11B illustrate how the myocardial performance profile and MPS can energetically phenotype patients into four unique profiles. Specifically, FIGS. 11A and 11B illustrate scattered plots depicting myocardial performance profile with respect to efficiency.

For example, in phenotype type A, generally depicted as area 1108 in FIG. 11B, Preserved Power Efficiency (MPS>1) has concordantly high API and CPO generally depicted as area 1108A correspondingly in FIG. 11A, and has the lowest clinical event rates. These patients have a low stress/strain relationship, and have coupled ventricles that are capable of efficient energy transfer. As a result, these have adequate mechanical and metabolic reserve.

For another example, in phenotype type B, generally depicted as area 1112 in FIG. 11B, Intermediate Power Efficiency with Preserved Mechanical Reserve (0.5<MPS<1) has a high CPO but low API generally depicted as area 1112A correspondingly in FIG. 11A, and has intermediate clinical event rates. The rate of change of potential energy outpaces the rate of change of power output. These patients are in a slow to moderately paced energetic deficit, which is being driven largely by a loss of mechanical efficiency. These patients have rightward shifted pressure-volume loops due to systemic hypervolemia with remodeled ventricles.

Similarly, in phenotype type C, depicted as area 1116 in FIG. 11B, Intermediate Power Efficiency with Reduced Mechanical Efficiency (0.5<MPS<1) has a low CPO but high API generally depicted as area 1116A correspondingly in FIG. 11A, and has intermediate clinical event rates. Similar to phenotype type B, these patients are in a slow to modestly paced energy depletion mode, while being driven a low power output. These patients tend to have more tall and narrow pressure volume loops with reduced stroke work, and can be seen in high afterload conditions and/or mixed shock states.

For yet another example, in phenotype type D, depicted as area 1120 in FIG. 11B, Low Power Efficiency (MPS<0.5) has a concordantly low API and CPO generally depicted as area 1120A correspondingly in FIG. 11A. Clinical event rates are the highest for this profile as it represents a ventricle with a high stress/strain relationship and an uncoupled ventricle with high energetic demand and low energy transfer efficiency. Finite metabolic reserves are rapidly depleted and patients are at risk for rapid disease progression.

Knowledge of the energetic profile such as those shown in FIG. 11B, as defined by the myocardial performance profile and MPS such as those shown in FIG. 11A, can help in identifying what is driving the patient's heart failure, and thus provide medical targets for disease stabilization. For example, as detailed hereinafter, a detection system including one or more sensors may be embedded or externally coupled or attached to a patient, such as in structural heart disease monitoring, a controller may be configured to be in data communication with the detection system to receive signals or data from the detection system. One or more parameters may be extracted from the signals or data received, and may be further processed by the controller. The parameters extracted or processed may include one or more of the mechanical efficiency, energetic states, potential energy, Ees, Ea, MPS, API, CPO, PAPI, SBP, DBP, SW, HR values, as discussed herein. The parameters extracted or processed may then be further visually presented in a display system. In some examples, the detection system may be remotely and/or wirelessly communicating with the controller. In some examples, the controller or the display system may be part of a portable device, and programs for extractions or processing may be a portion of a portable device app. In this regard, the portable device app may visually inform a user, for example, the patient or a clinician a stratification of the health of the patient, a prognosis of the failure, and healthcare, clinical supports, or provocative maneuvers needed to be prescribed dynamically, or in near real-time. For example, the portable device app may visually or audibly animate one or more of the stratification, prognosis, or healthcare, clinical supports, or provocative maneuvers needed, with texts, colors, gauges, alarms, vibrations, and the like. In some examples, the portable device app may further communicate with a delivery system to carry out the healthcare, clinical supports, therapy guides, or provocative maneuvers needed via the portable device app, while the patient is being monitored. In other examples, the portable device app may further communicate with a hospital, a care team, a clinician, a nurse station, to further inform them the patient's conditions detected.

Similarly, FIG. 11C shows heart failure predictions 1160 derived from CPO and API values. Specifically, FIG. 11C portion A 1162 illustrates LVAD or Transplant free rate based on CPO and API values across time. FIG. 11C portion B 1164 illustrates pressure changes with respect to volume changes. More specifically, portion B 1164 exemplarily illustrates how API may be derived from measurable or extractable parameters such as systolic blood pressure (SBP), diastolic blood pressure (DBP), and pulmonary capillary wedge pressure (PCWP), and how CPO may be derived from other measurable or extractable parameters such as stroke work (SW) and heart rate (HR). FIG. 11C portion C 1164 illustrates how relationships between API and CPO may affect clinical even rate. For example, graph 1166 illustrates a positive concordant relationship between API and CPO with both API and CPO being informative, which then accounts for a low clinical event rate. Graph 1168 illustrates a discordant relationship between API and CPO with API being more informative than CPO which is less informative, which may then account for a remodeled LV (LV dilation to preserve stroke volume), or systematic volume overload (non-cardiac volume retention). Graph 1170 illustrates a discordant relationship between API and CPO with CPO being more informative than API which is less informative, which may then account for an intermediate clinical event rate, with mixed shock or stiff aorta. Graph 1172 illustrates a negative concordant relationship between API and CPO with both API and CPO being informative, which then may account for a high clinical event rate.

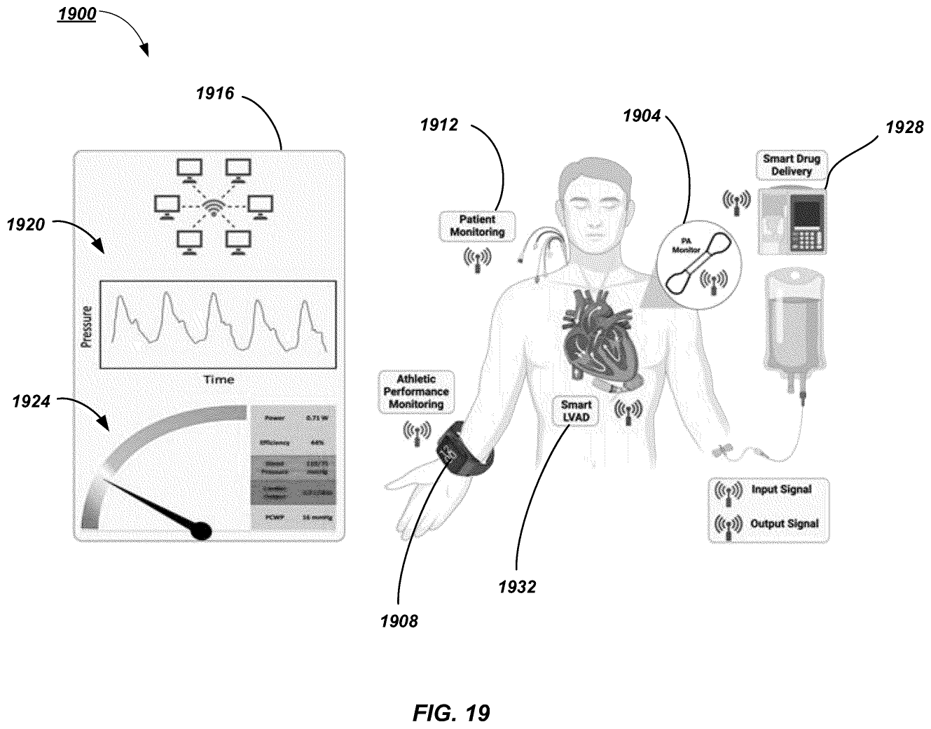

Similarly, FIG. 11D shows an exemplary advanced hemodynamics 1180 for different stages of risk stratification. For example, first stage 1182 illustrates an uncoupled ventricle with poor reserve based on less informative parameters. Second stage 1184 illustrates an uncoupled ventricle with adequate reserve. Third stage 1186 illustrates a dynamic recoupling of ventricle, and fourth stage 1188 illustrates a persistently coupled ventricle with adequate reserve. The display of one or more of the different stages of risk stratification may be based on one or more of the analysis described in FIGS. 11A-11C. For example, the risk stratification may be based on myocardial profiles and/or efficiency maps such as described above. Although four stages are pictured, more or fewer stages may be included. In some examples the different stages may be displayed as icons on a display screen or one or more graphical user-interfaces. In some examples, after data is collected and an analysis is performed, one stage may be displayed. For example, first stage 1182 may be displayed for a user to quickly and efficiently view a current stage of risk stratification for a particular patient. In some examples, one or more stages of risk stratification may be displayed on a display along with other data. When one or more of the risk stratification stages are displayed along with other data, the icons may be sized and/or otherwise modified in order to fit the display screen along with other data. For example, the icons may be sized to fit a mobile display and/or a display associated with a controller device such as described below with regards to FIG. 19.

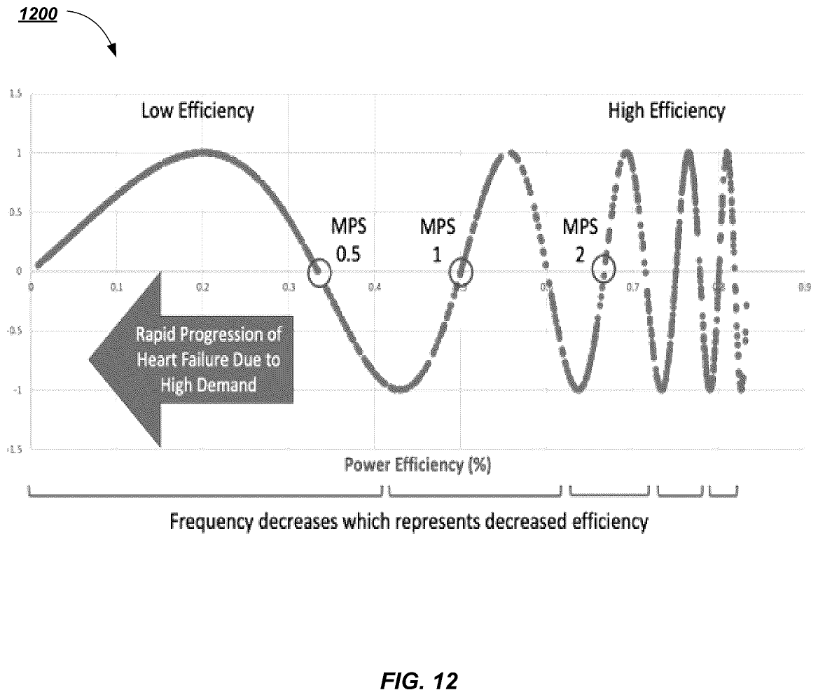

FIG. 12 illustrates a sine [sin(2πMPS)]transformation 1200 of the MPS to remove the time domain. When the transformation is plotted against P_{Eff}, FIG. 12 also shows rapid progression of heart failure due to high demand. The frequency is inversely proportional to efficiency. As the frequency increases, efficiency goes down. The greatest rate of change of loss of efficiency occurs at an MPS of 0.5.

### API, CPO and MPS in the Interpretation of the Starling Curve

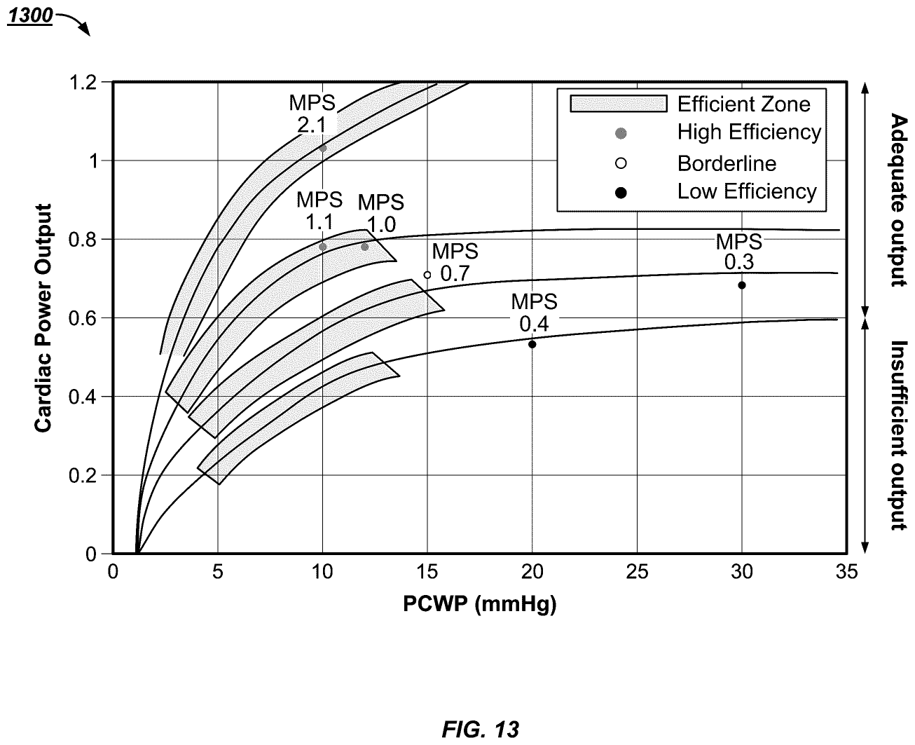

FIG. 13 illustrates an interpretation 1300 of the Starling curve in the context of power output, efficiency and MPS. If a Starling curve is created with PCWP as the input variable (x-axis) and CPO as the output variable (y-axis), an ideal metabolic performance score for the patient may be defined. The more rightward shifted one is on a particular Starling curve, the lower the API and Eff and the more uncoupled the ventricle. The zone of maximal efficiency for a given starling curve is denoted by the inflection point of the Starling curve and is a true physiologic threshold for a patient. Here the MPS is the height (CPO) and degree of right shift (API) on a particular starling curve. Patients with an MPS of <0.5 are operating on the flat part of their respective starling curve and the ventricle is uncoupled to the downstream circulation. When MPS is 0.5 to 1, the patient is still in a modest energy depletion mode with a higher potential energy than power output and when the MPS is >1 the patient is operating in a high efficiency zone.

### Harvi Modeling and Practical Application of the Myocardial Performance Profile and Myocardial Performance Score

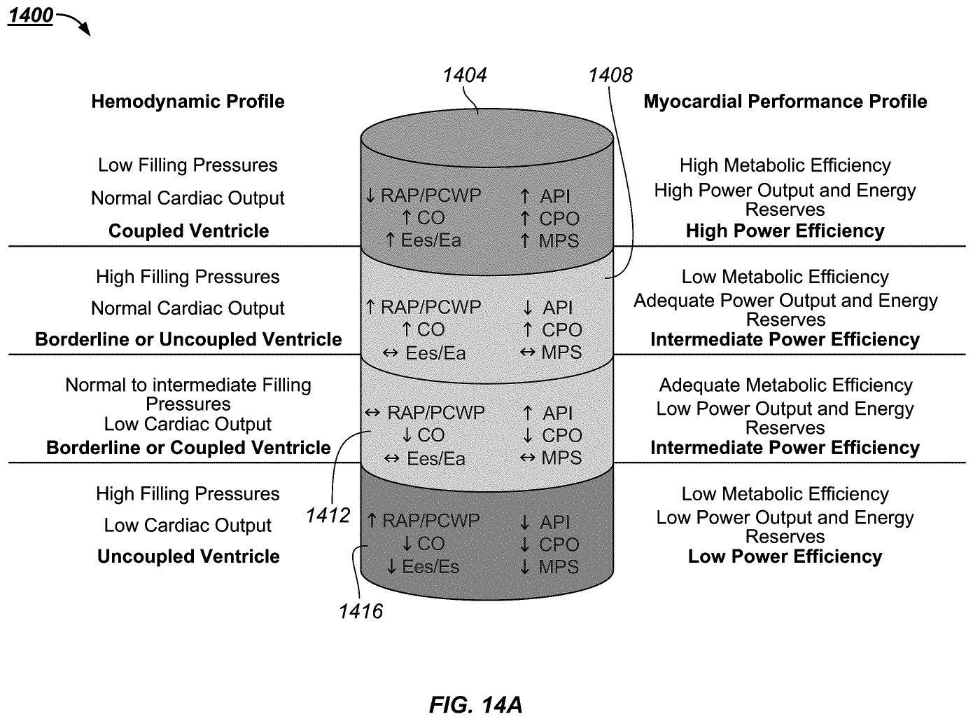

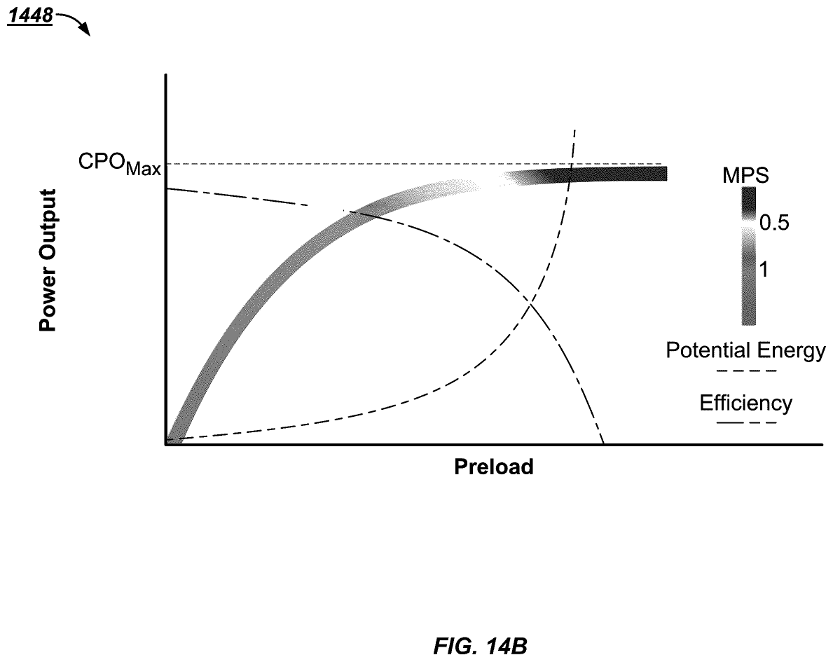

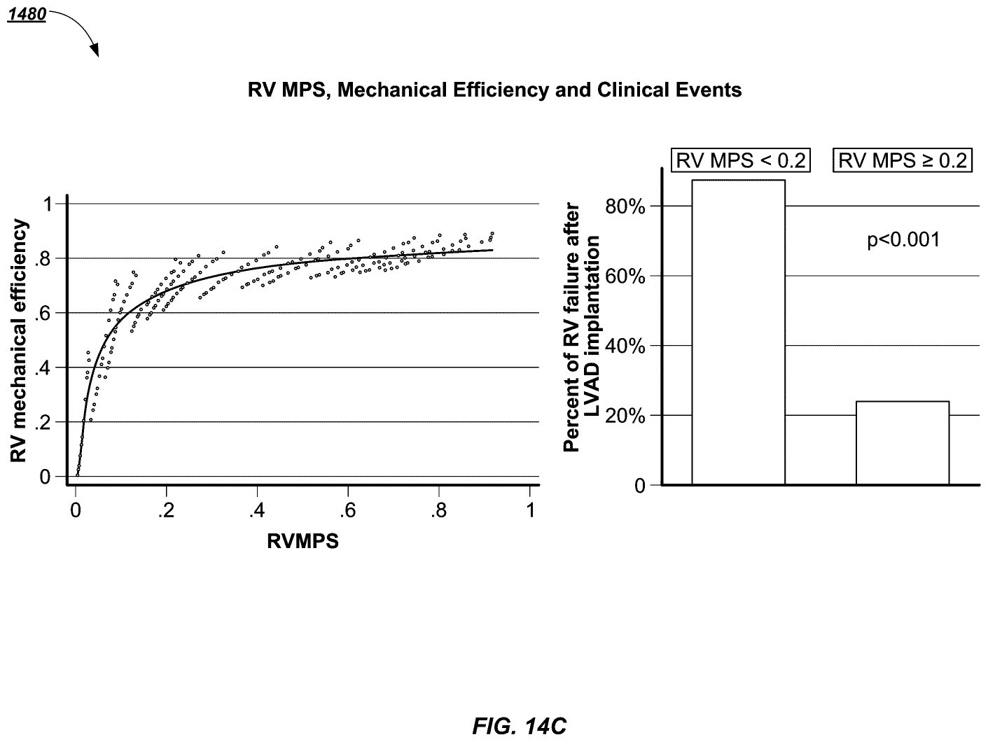

FIG. 14 illustrates a comparison 1400 of the hemodynamic profile to the myocardial performance profile. Like the hemodynamic profile which classifies patients based on their perfusion state (cardiac output) and congestive state (PCWP), a myocardial performance profile can be established which classifies patients based on their power output (CPO) and efficiency of energy handling (API/PAPI). As discussed above, an MPS of 1 represents the point where power output and potential energy are equal. An MPS of 2 is the point where power is twice potential energy and an MPS of 0.5 is when potential energy is twice the power output. The Peff at an MPS of 0.5 is thus 33.33%, the Peff at an MPS of 1 is 50% and the Peff at an MPS of 2 is 66.67%. The top tier 1404 represents an MPS>1, at this point power is greater than potential energy, efficiency is high, the patient is in a favorable energetic state. In the middle tiers 1408, 1412, the MPS score is between 0.5 and 1, which may mean either efficiency is low, or power is low, and potential energy is greater than power output. The patient is consuming finite energy stores at an increased rate but not as much as in bottom tier 1416. In the bottom tier 1416, the MPS score is less than 0.5, which may represent a true tipping point in which efficiency rapidly drops because potential energy dramatically increases. This patient is rapidly consuming finite energy stores and is the sickest. FIG. 14B generally illustrates a relationship 1448 between MPS, power, efficiency, and potential energy. Potential energy rises expeditiously at an MPS score of 0.5 with a concomitant drop in efficiency. Further power output is limited at this point and power plateaus. Similarly, the energetic tipping point of the right ventricle can be defined using the RV-MPS. For example, FIG. 14C illustrates a precipitous drop 1480 in mechanical efficiency when the RV-MPS is less than 0.2.

By knowing the simultaneous CPO and API/PAPI, a patient's exact x, y coordinates and efficiency of energy handling (from the slope and power efficiency to MPS relationship) can be determined from the Starling curve. These coordinates define the myocardial performance profile for the patient and supply valuable prognostic information for a patient.

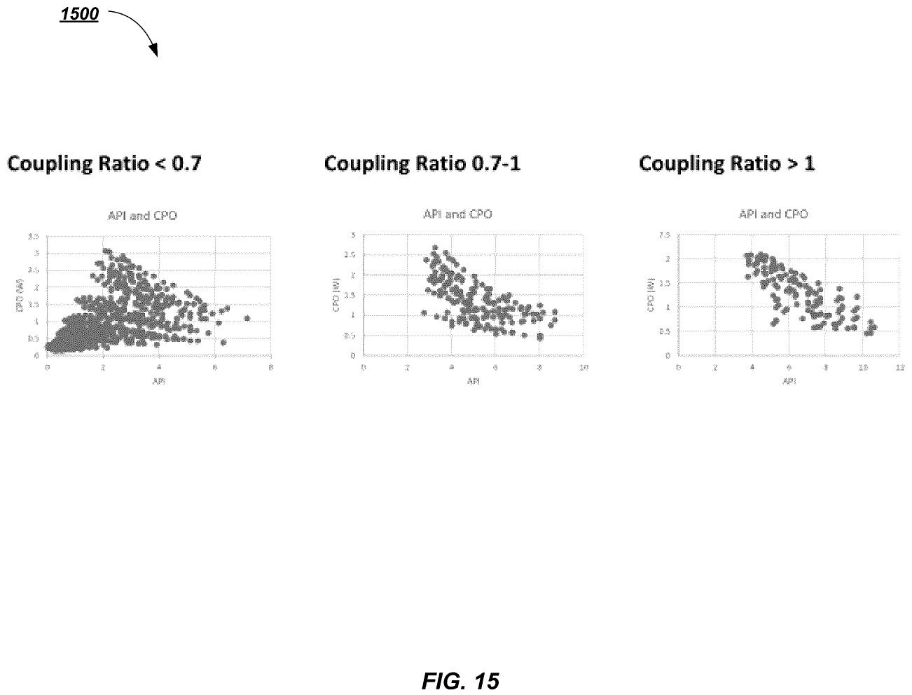

FIG. 15 illustrates relationships 1500 of API and CPO to coupling ratios (e.g. VACs). Using Harvi, over 1010 patient scenarios of patients with varying degrees of heart performance were created by varying Ees, Ea, blood volume, and HR and recording the hemodynamic output. The scenarios were then sorted based on the coupling ratio. 126 of the 1010 scenarios established a coupling ratio >1 (fully coupled), 167 scenarios returned a coupling ratio of 0.7 to 1 (borderline coupled) and 718 scenarios returned a coupling ratio <0.7 (uncoupled). In the coupled group, all patients had a PCWP≤20 mmHg and API>3. A CPO<0.6 W occurred in 12 patients (9.5%), and all were associated with high output, low vascular tone states being driven by low blood pressure. In the borderline coupled group, 159 of 167 scenarios (95.2%) had a PCWP≤20 (all cases ≤23 mmHg) and all patients had an API>2.5. A CPO<0.6 W occurred in 7 of 167 patients (4.2%). In contrast, in the uncoupled scenarios 392 of 718 scenarios (54.6%) had a PCWP>20 mmHg and 307 of 718 scenarios had a CPO<0.6 W (42.8%). When the uncoupled group was restricted to those with a PCWP>20 mmHg and CPO<0.6 W (228 scenarios), all patients had an API<2. Under these conditions, API was able to predict the coupling ratio with an R² of 0.945. See also FIG. 1B.

In summary, categorizing a patient according to their power output (high: CPO≥0.6 W; low: CPO<0.6 W) and mechanical efficiency (high: API≥2.5; low: API<2) or with the combined entity of the MPS (high power efficiency: MPS>1, low power efficiency MPS<0.5) can help with additional risk stratification and prognostication.

### Clinical Validation

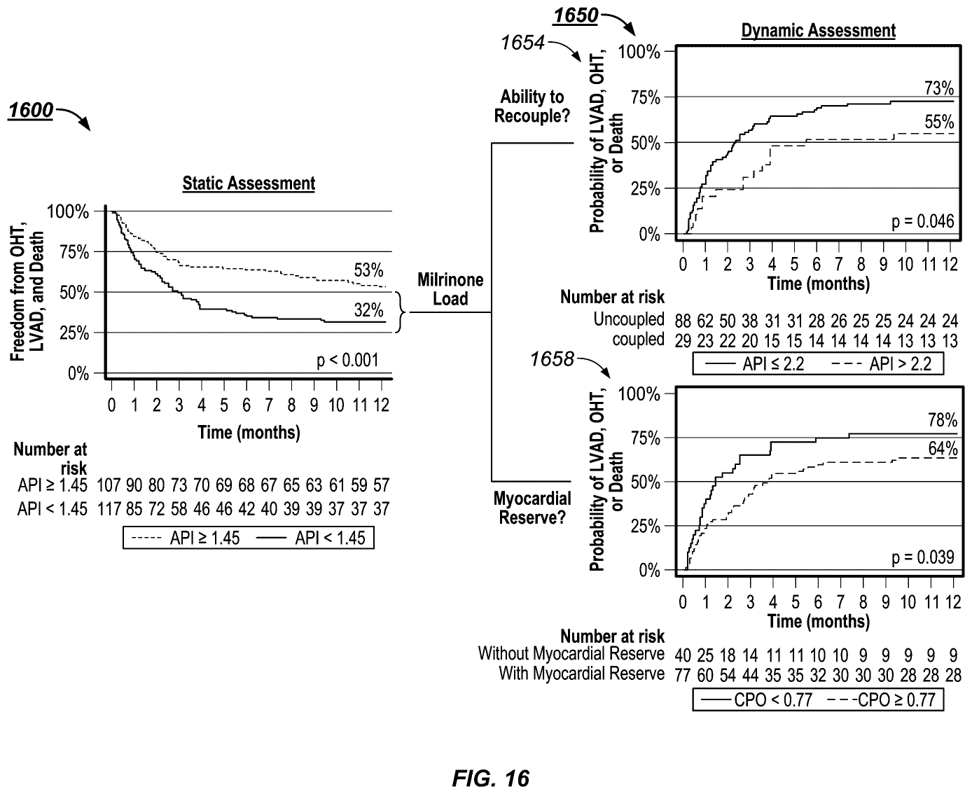

FIG. 16 illustrates static assessments 1600 and dynamic assessments 1650 of the prognostic role of API and/or CPO to predict one year survival free from left ventricular assist device (LVAD) or orthotopic heart transplantation (OHT), or transplant. A cohort of 224 patients with AHA/ACC Stage D heart failure who were presented with shock to the cardiac catheterization laboratory with initial hemodynamics warranting inotropic infusion were studied as part of a milrinone database. A detailed description of this patient cohort has previously been described. Baseline and final API and CPO were able to add additional risk stratification by phenotyping patients by their ability to recouple (improve efficiency) 1654 or generate sufficient contractile reserve (augmentation in CPO) 1658 with milrinone infusion.

FIG. 16 also illustrates show that provocative maneuvers in the form of drug challenges (i.e. milrinone, dobutamine, nitroprusside) or ventricular unloading with mechanical devices can further clarify the energetic profile and risk profile by defining whether or not a patient with an initial low or intermediate power efficacy profile ascertained from a static hemodynamic assessment have the ability to recouple their ventricle and/or augment mechanical reserve sufficiently to return to a more favorable energetic profile.

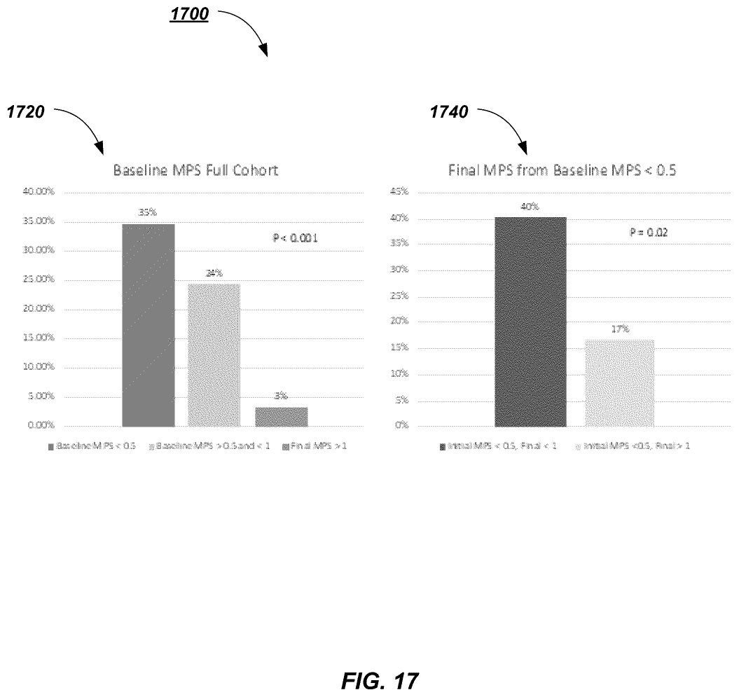

FIG. 17 illustrates a risk stratification 1700 based on initial MPS 1720 and final MPS 1740 after milrinone infusion. The MPS score was able to add additional risk stratification. Patients with a baseline MPS of <0.5 had a 35% rate of death, LVAD or transplant at 30 days whereas those with an initial MPS<1 had only a 3% rate of the combined endpoint. When the sickest cohort was reexamined after milrinone infusion, those able to augment their MPS from an initial score of <0.5 to a final score >1 had a better prognosis than those who could not.

The efficient use of finite energy reserves is of the utmost importance to promote recovery and/or stabilization. A ventricle that is coupled to the downstream circulation is capable of efficiently using energy stores. The ventriculo-arterial unit is said to be coupled when the contractile ability of the ventricle (end-systolic elastance, Ees) is matched by the ability of the circulation to accept the ejected blood which is decided by elastance of the vasculature (effective arterial elastance, Ea). In unstressed conditions with normal ventricular function, Ees is greater than Ea with an Ees/Ea (coupling ratio) of 1.5 to 2.0 which maximizes mechanical efficiency. As ventricular function worsens (decrease in Ees), the heart sacrifices efficiency to maintain adequate cardiac output and tissue perfusion. Activation of the renin-angiotensin-aldosterone (RAAS) system leads to retention of salt and water increasing effective circulatory and ventricular preload. RAAS activation also causes arteriole vasoconstriction which increases vascular tone and Ea. As a result of this remodeling process, Ees/Ea decreases, the ventricle shifts downward and rightward on the pressure-volume (PV) curve and tends to operate on the flatter part of the Starling curve. By doing so the heart can maximize output but it comes at the expense of efficient energy handling.

It is shown that the physiologic inflection points when the efficiency of energy transfer exponentially falls can be predicted using the simultaneous assessment of API and CPO which can be better understood by incorporating these two complex variables into a single entity called the MPS. As input power increases, over-stretching of the action-myosin filaments occurs leading to inefficient crossbridge formation and excess crossbridge heat. Concurrently, the collagen and elastin components of the vasculature are also stretched leading to a decrease in vascular compliance. When these changes occur, the rate of change of potential energy (which in turn reflects excess heat production at a cellular level from inefficient cross-bridging and from wavelet reflections in the aorta impeding antegrade flow), outpaces power output and the system efficiency drops rapidly. This occurs when the MPS is <0.5. This is the point of maximal rate of change of efficiency and is the terminal stages of heart failure. The system is perfectly balanced in terms of power output and efficiency when the MPS is 1. At this point, the API is 2/CPO.

At the limits of heart stretch, heat is generated out of proportion to power production (potential energy increases out of proportion to kinetic energy). Since energy for the system is conserved, as potential energy increases, kinetic energy must drop. For the cardiovascular system, API reflects the efficiency of energy transfer and is inversely proportional to the potential energy of the system. Thus, as API decreases, potential energy increases and linear kinetic energy decreases leading to a reduction in the rate of change of cardiac output. Thus, MPS represents the limits of the kinetic energy/potential energy relationship and reflects the limits of stretch on the system. The MPS represents cardiovascular performance relative to a theoretical maximal performance defined by the limits of output and efficiency for the ventriculo-arterial unit.

### EXAMPLES

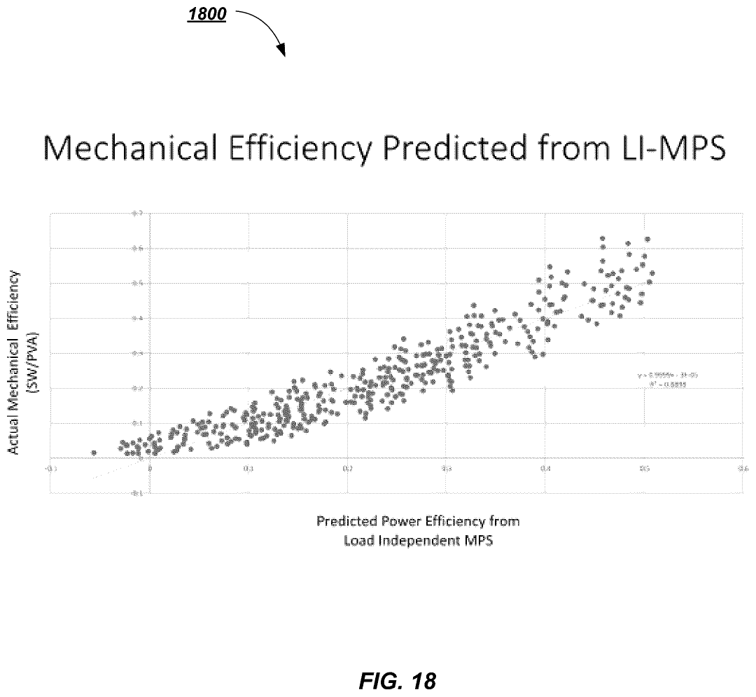

The relationship 1800 between power efficiency and MPS is independent of input as shown in FIG. 18, which illustrates mechanical efficiency predicted from load independent MPS.

The input-independent nature means that assessments of the ventricular-arterial unit can be made even without knowing the loading conditions of the heart. This means that prognostic information about performance can be obtained by analyzing the pressure vs time tracing in any vascular system since the components that make up the load-independent relationship can all be obtained non-invasively from waveform analysis (cardiac output, MAP and PP can all be obtained from pulse wave contour analysis). Thus, this proof serves as the basis for a non-invasive analysis of overall performance that can be linked to any peripheral device that is able to transmit a pressure vs time arterial signal (i.e., wearable watches or internal or external pressure monitors). The algorithms can be built into proprietary implantable or wearable devices, or they can be implemented as a system on a chip that allows for integration into existing wearable or implantable pressure measuring devices.

### Real-Time Patient Monitoring, Smart Drug Delivery System and Smart VADs

By performing pulse wave contour analysis of either a temporary or implantable pulmonary artery pressure vs time signal (i.e., from a continuous Swan Ganz catheter for patients in the hospital or from an implantable PA sensor for ambulatory patients), right ventricular power and efficiency can be determined. By back calculating, an estimated RA pressure can also be obtained. Similarly, pulse wave contour analysis from a wearable watch or other peripherals which can measure systemic pressure vs time signals or implantable systemic arterial pressure devices would supply information on global and left ventricular power and efficiency. Similarly, an estimated PCWP or LA pressure can also be obtained from back calculations. These parameters can be tracked over time for overall risk assessment and can serve as a more objective assessment of global risk for a patient than current parameters. Alerts could be built into the system to inform the patient and/or provider when power or efficiency drops to allow for an intervention.

A closed loop system is shown in FIG. 19 where signal, data, or information from an implantable hemodynamic monitoring device is directly sent to a second hardware device such as a computerized ambulatory delivery device (CADD) pump for drug delivery or a ventricular assist device (VAD) to allow for real-time modulation of drug infusion rate and/or concentration as well as real-time modulation for VAD speed. Current CADD delivery systems for parenteral vasoactive medications provide drug delivery at a fixed rate. Similarly, current LVADs provide a set speed with periodic oscillations in the speed which are completely independent from the patient. By incorporating the MPS algorithm, a smart drug delivery system would be able to modulate drug infusion rate based on real-time efficiency and power needs. During rest, power needs are less than during peak exercise performance and the smart CADD device would accommodate with a reduced infusion rate. Similarly, a smart VAD would modulate its rotational speed to maximize efficiency of the right ventricle and/or left ventricle depending on patient-specific needs. Ideal flow could be set or determined for the patient to maximize ventricular efficiency to aid with recovery when performance may be unnecessary (sleep, day to day activity) but would change the flow rate to maximize ventricular power when peak performance is desired (exercise). Additional inputs such as targeting aortic valve opening with at least intermittent native aortic valve flow as well as a targeted left ventricular diastolic pressure can further refine the LVAD settings. In this capacity, the mechanical pump (VAD) or smart drug delivery system would be coupled to the human pump (heart) to maximize overall performance of the pump-patient continuum.

FIG. 19 illustrates an exemplary diagram showing a monitoring system 1900. The monitoring system 1900 may include timed input data or signals from one or more of a PA catheter, an implantable PA monitor 1904, implantable arterial pressure monitors 1906, or percutaneous devices 1908 such as smart watches capable of obtaining a pressure, and a patient monitoring device 1912 such as an arterial pressure monitoring device. The input signals may be collected remotely or wirelessly or wired to a controller device 1916. The controller device 1916 may further include a processor that processes the collected input signals, and a plurality of display devices such as a pressure display 1920 and a power efficiency surrogate meter 1924, as non-limiting examples. Additionally, or alternatively, the controller device 1916 may analyze or process the input signals collected based on one or more executable codes or programs, categorize the signals collected, and display the signals categorized as one or more of the risk stratifications of the advanced hemodynamics 1180 of FIG. 11D (e.g., first stage 1182, second stage 1184, third stage 1186, fourth stage 1188 in order to efficiently provide a visual analysis or stratification to a user based on the input signals collected.

For example, pressure wave contour analysis may be used to calculate the power efficiency surrogate and MPS score, which can provide information, data, or output signals, on overall cardiac performance. Additionally or alternatively, the power efficiency surrogate and/or the MPS score may be recorded, analyzed, and displayed on the display devices. Further, some or all the overall cardiac performance, the power efficiency surrogate, the MPS score, and the output signals may be transmitted to further assist care of one or more patients. For example, the output signals can be transmitted to either a drug delivery system 1928 of ventricular assist device to modulate drug delivery or device speed to improve cardiac performance.

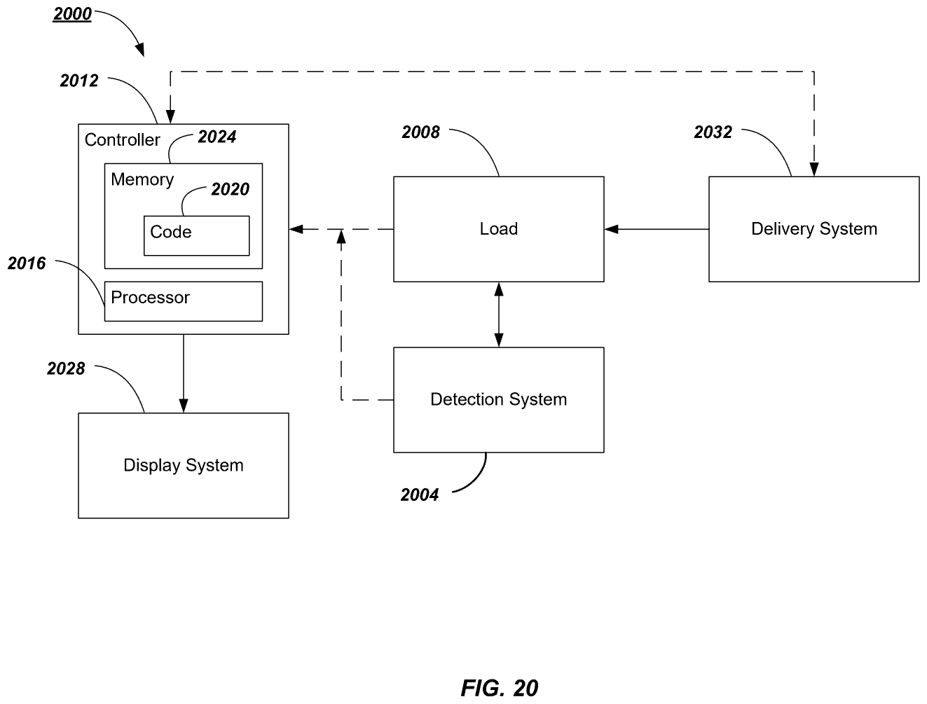

FIG. 20 illustrates an exemplary example of a generalized monitoring system 2000. The generalized monitoring system 2000 may include a sensing or detection system 2004. In some examples, the detection system 2004 may include at least one of a PA catheter, an implantable monitor for PA, implantable arterial pressure sensors or monitors, percutaneous devices, and/or a patient monitoring device. In some examples, the implantable monitor for PA is implanted in a load 2008 such as the patient of FIG. 19 to detect parameters internal to the load 2008. Signals generated, sensed, or detected by the detection system 2004 are generally configured to be transmitted from the detection system 2004 to a controller 2012. In some examples, the controller 2012 may ping or collect the signals periodically or continuously from the detection system 2004. The controller 2012 may be coupled to the load 2008 and the detection system 2004 wirelessly and may be located remotely from the load 2008 and the detection system 2004. In other examples, the controller 2012 may be hardwired to the load 2008 and the detection system 2004.

In some examples, the controller 2012 may also include a processor 2016 that processes and analyzes the signals collected from the load 2008 and the detection system 2004, based on some or all sequences of codes, instructions, or programs 2020 stored in a memory 2024. In some examples, the controller 2012 or the memory 2024 are in a remote server (not shown), whereas the processor 2016 may be located separately from the remote server. In some examples, the controller 2012 may be housed in a moveable housing (not shown) for portability purposes. In some examples, the processor 2016 may analyze the signals obtained from the detection system 2004, and then continuously or periodically analyze the signals obtained based on settings provided by a user.

In some implementations, server computers may not be necessary and/or preferred. For example, in one or more implementations, the controller 2012 can implement one or more aspects of the present disclosure. In some other examples, multiple detection system 2004 may be connected to networks implemented with one or more of the different server computers.

In some implementations, the generalized monitoring system 2000 may include a display device 2028 configured to display some or all the analyzed signals. The display device 2028 may include a high-resolution liquid crystal display (LCD), plasma, light emitting diode (LED), or organic light emitting diode (OLED) panel which may be flat or curved as shown, a cathode ray tube, or other conventional electronically controlled video monitor. In some examples, the analyzed signals may include cardiac pressure, power efficiency, MPS, etc., which may be detected continuously or periodically. In some examples, the analyzed signals may be detected based on a user input. In some other examples, the analyzed signals may be detected based on preset or default settings. In some examples, some or all the analyzed signals may cause the processor 2016 to adjust a delivery system 2032. In some implementations, the delivery system 2032 may be embedded in the load 2008 for internal delivery of drug to meet a specific or predetermined load parameter. For example, the controller 2012 generates a number of output signals to affect how the delivery system 2032 controls the amount of drugs to be delivered to the load 2008. In one implementation, the output signals may affect how an implanted ventricular assist device modulates drug delivery to improve cardiac performance based on any feedback received from the controller 2012. In another implementation, the output signals may affect how a smart delivery device delivers a specific kind of drug to improve cardiac performance based on the output signal(s) received from the controller 2012.

### Periprocedural Energetic Modeling for Structural Heart Disease and Cardiac Device Interventions

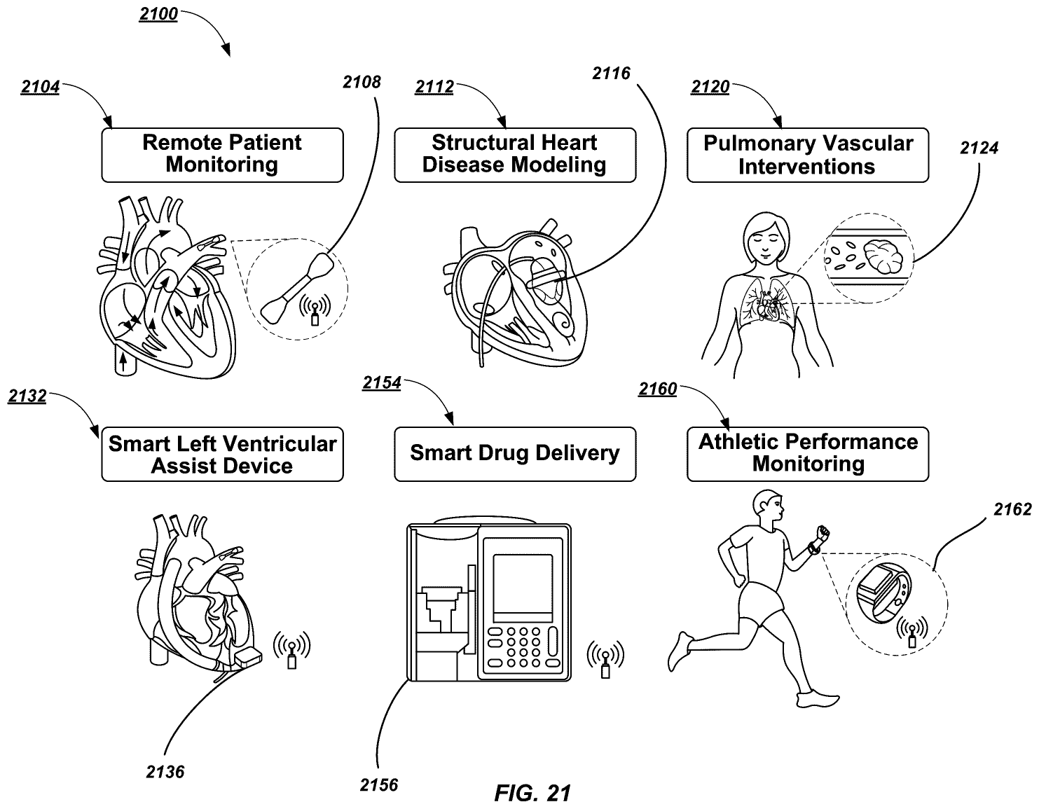

FIG. 21 illustrates different exemplary applications of a general monitoring system 2100. For example, FIG. 21 illustrates a remote patient monitoring system 2104 having an implanted monitoring device 2108 embedded in a heart, or between the heart and the lung. In some examples, the implanted monitoring device 2108, such as CardioMems, a sensor that monitors heart failure by measuring pulmonary artery pressure and heart rate, an artery sensor, or a PA sensor, may be configured to periodically or continuously detect or measure heart failure rate with some or all of the parameters mentioned above, and transmit the results or parameters measured to the monitoring system 1900. The remote patient monitoring system 2104 may be able to pinpoint specific remedy or care needed to be provided to the patient. FIG. 21 also illustrates a structural heart disease modeling system 2112 to determine if the patient may be benefited from an implanted device 2116, such as a Mitraclip. For example, the structural heart disease modeling system 2112 may accurately assess if a patient will be benefitted from or a good candidate for a structural implant based on the hemodynamics and the phenotypes as discussed, before a heart procedure is to commence. Similarly, FIG. 21 also illustrates a pulmonary vascular intervention application 2120 for the right side of the heart, and a smart left ventricular assist device 2132. More specifically, the pulmonary vascular intervention application 2120 focuses primarily on right ventricular diseases such as pulmonary aneurism (blood clots in the lungs) and pulmonary artery hypertensions, and thus therapies, based on right ventricular energetics individually tailored for a specific patient, as opposed to a general threshold for all patients and all age groups.

At a pathophysiologic level, these devices improve the neurohormonal axis, i.e. Baroreceptor stimulation (or Barostim), improve ventricular and valvular mechanics (i.e. mitral valve interventions, tricuspid valve interventions, aortic valve interventions) or improve ventricular efficiency and energetics (i.e. cardiac contractility modulation and cardiac resynchronization therapy). Defining who will benefit from these technologies is foundational to disease stabilization and improving care delivery.

For valvular interventions, underlying myocardial energetic reserve is a pre-requisite as patients with limited reserve often do not hemodynamically tolerate valvular interventions. Current practice uses structural endpoints such as ventricular cavity size or volume or annulus size to help guide eligibility for valvular interventions. In reality, these structural endpoints are a surrogate for the underlying ventricular energetics and mechanical reserve. Ventricular energetic modeling as described above, prior to a planned valvular intervention, can provide a more nuanced assessment of the patient and can help guide the appropriate intervention. For neuro-modulatory devices and devices that improve LV contractility or resynchronization, energetic modeling can help identify patients who are most likely to respond from these interventions (hyper-responders).

### Right Ventricular Performance Monitoring in Pulmonary Hypertension and Pulmonary Embolism Therapy

The RV-MPS and overall knowledge of right ventricular performance using the complete hemodynamic and energetic profile can also be used to guide interventions targeting the pulmonic vasculature. Pulmonary vasodilator therapy, both oral and parenteral, are the mainstay of therapy for patients with pulmonary hypertension. The RV-MPS can be used to guide indications for pulmonary vasodilators and response to therapy. A targeted RV-MPS can be used to escalate and de-escalate pulmonary vasodilator therapy, thereby maximizing efficacy and minimizing side-effects. Similarly, for patients who develop pulmonary embolisms, thrombolytic therapy and/or mechanical clot extraction is often indicated. The continuous monitoring of the RV-MPS and right ventricular energetic profile can be used to guide duration of thrombolytic therapy and response to treatment. FIG. 21 shows an exemplary smart drug delivery system 2154 having a communication device 2156 that may be wirelessly coupled to some or all of the monitoring systems, such as the monitoring system 1900 of FIG. 19, to remotely request for a drug delivery. In some examples, the exemplary smart drug delivery system 2154 may control a delivery of drug, for example, an appropriate concentration of drugs derived from the energetics detected, to the patient dynamically or in near real-time. For example, when a patient is resting, her energetic parameters may be different when the patient is active, thus a smart drug delivery system may accurately provide specific treatment or care to the patient, and may reduce possible side effects of the drugs administered.

### Athletic Performance Tracker and Heart Failure Performance Tracker

FIG. 21 also shows an athletic performance monitoring system 2160 having a wearable device 2164. The MPS and the power efficiency surrogate represent the theoretical limits of myocardial performance as they are indicative of limits of stretch and potential energy for the ventriculo-arterial unit. Thus, an individual's performance is reported as a percentage of a theoretical maximum performance. Athletic performance (peak power output during exercise, pulse pressure variation, dynamic effective arterial elastance (PP/SV) and overall efficiency) can be derived from pulse wave contour analysis from the percutaneous devices 1908 such as a wearable watch or other peripheral with pressure monitoring capabilities. An individual or a programmed device would then be able to monitor or process their progress over time during training regimens. A similar analysis can be done for patients with heart failure. Overall performance can be monitored during cardiac rehab or day to day activities of daily living to track progress over time.

It is important to note that the MPS and P_{Eff} surrogate are highly accurate when an individual is approaching the limits of stress/strain of the myofilaments and vasculature. Most heart failure patients live in this range both at rest and with exercise. Healthy athletes will not fall in this range at rest but with sufficient effort, should enter this range with peak exercise. Thus, this monitoring would be relevant during peak exercise for healthy individuals.

It will be understood that all or part of one or more of FIGS. 1-21 may also depict novel, ornamental, non-functional features of the monitoring system, e.g., some in the form of ornamental features of graphical user interfaces (GUIs). These novel, ornamental features may be separate from and capable of existing independently from the functional features described and illustrated herein.

While the disclosure has been described with respect to the figures, it will be appreciated that many modifications and changes may be made by those skilled in the art without departing from the spirit of the disclosure. Any variation and derivation from the above description and figures are included in the scope of the present disclosure as defined by the claims.

What is claimed is:

1. A cardiac monitoring control system comprising:
a display device;
a detection system configured to periodically detect one or more parameters at a load;
a delivery system coupled to the load, and operable to deliver one or more treatments to the load; and
a controller, coupled to the display device and the detection system, comprising a processor and a memory storing instructions, which, when executed, cause the processor to at least:
receive signals indicative of the one or more parameters periodically detected at the load transmitted by the detection system,
iteratively generate a user-specific ventriculo-arterial coupling (VAC) ratio based on the signals received,
control the display device to animate the VAC ratio dynamically,
activate the delivery system in response to the VAC ratio,
transmit activating instructions, based on the VAC ratio, to the delivery system coupled to the load, and
control the delivery system to deliver a user-specific treatment updated dynamically based on the VAC ratio.

2. The cardiac monitoring control system of claim 1, wherein the delivery system further comprises at least one of a computerized ambulatory delivery device (CADD) pump to deliver a drug delivery, and a ventricular assist device (VAD).

3. The cardiac monitoring control system of claim 1, wherein the user-specific treatment includes at least one of a modeling of the load, and an intervention for the load.

4. The cardiac monitoring control system of claim 1, wherein the detection system utilizes at least one pulmonary artery catheter to measure the one or more parameters of the load.

5. The cardiac monitoring control system of claim 1, wherein the instructions, when executed, cause the processor to recognize at least one of a phenotype, a power efficiency, a preserved mechanical reserve, and a clinical event based on the one or more parameters, and to transmit information to the display device based upon at least one of the phenotype, the power efficiency, the preserved mechanical reserve, and the clinical event recognized.

6. The cardiac monitoring control system of claim 1, wherein the instructions, when executed, cause the processor to analyze the signals to generate data indicative of a user-specific myocardial performance (MPS) score.

7. The cardiac monitoring control system of claim 6, wherein the instructions, when executed, cause the processor to generate data indicative of a first alert at the display device when the user-specific MPS score drops below a first threshold, and a second alert at the display device when the user-specific MPS score drops below a second threshold that is below the first threshold.

8. A method of delivering a user-specific treatment in a cardiac monitoring control system that comprises a display device, a detection system operable to periodically detect a plurality of parameters at a load, a delivery system coupled to the load, and operable to deliver one or more treatments to the load, the method comprising:
transmitting signals indicative of the plurality of parameters periodically detected at the load;
generating data indicative of a user-specific ventriculo-arterial coupling (VAC) ratio iteratively based on the signals transmitted;
animating on the display device the VAC ratio dynamically;
activating the delivery system based on the VAC ratio; and
transmitting data indicative of the user-specific treatment updated dynamically based on the VAC ratio to the delivery system.

9. The method of claim 8, wherein the delivery system further comprises at least one of a computerized ambulatory delivery device (CADD) pump to deliver one or more drugs, and a ventricular assist device (VAD).

10. The method of claim 8, wherein the user-specific treatment includes at least one of a modeling of the load, and an intervention for the load.

11. The method of claim 8, further comprising detecting the parameters of the load with a pulmonary artery catheter in the detection system.

12. The method of claim 8, further comprising:
recognizing at least one of a phenotype, a power efficiency, a preserved mechanical reserve, and a clinical event based on the plurality of parameters; and
transmitting information to the display device based upon at least one of the phenotype, the power efficiency, the preserved mechanical reserve, and the clinical event recognized.

13. The method of claim 8, further comprising generating data indicative of a user-specific myocardial performance (MPS) score based on the signals.

14. A non-transitory computer-readable medium storing a plurality of instructions for use with a cardiac monitoring control system for generating a user-specific treatment, the cardiac monitoring control system comprising a display device, a detection system configured to periodically detect a parameter at a load, a delivery system coupled to the load, and operable to deliver one or more treatments to the load, and a controller, coupled to the display device and the detection system, the instructions, when executed, cause the controller to perform the steps of:
generating signals indicative of the parameters periodically detected at the load detected by the detection system;
iteratively animating, at the display device, a user-specific ventriculo-arterial coupling (VAC) ratio based on the signals received; and
activating the delivery system in response to the VAC ratio to deliver the user-specific treatment updated dynamically based on the VAC ratio.

15. The non-transitory computer-readable medium of claim 14, wherein the delivery system further comprises at least one of a computerized ambulatory delivery device (CADD) pump to deliver a drug delivery, and a ventricular assist device (VAD).

16. The non-transitory computer-readable medium of claim 14, wherein the user-specific treatment includes at least one of a modeling of the load, and an intervention for the load.

17. The non-transitory computer-readable medium of claim 14, wherein the detection system utilizes at least one pulmonary artery catheter to measure the parameter of the load.

18. The non-transitory computer-readable medium of claim 14, wherein the instructions, when executed, cause the controller to perform the steps of recognizing at least one of a phenotype, a power efficiency, a preserved mechanical reserve, and a clinical event based on the parameter, and transmitting information to the display device based upon at least one of the phenotype, the power efficiency, the preserved mechanical reserve, and the clinical event recognized.

19. The non-transitory computer-readable medium of claim 14, wherein the instructions, when executed, cause the controller to perform the steps of generating data indicative of a user-specific myocardial performance (MPS) score based on the signals.

20. The non-transitory computer-readable medium of claim 19, wherein the instructions, when executed, cause the controller to perform the step of generating data indicative of a first alert at the display device when the user-specific MPS score drops below a first threshold, and a second alert at the display device when the user-specific MPS score drops below a second threshold that is below the first threshold.
